(19) United States
(12) Patent Application Publication
(10) Pub. No.: US 2026/0041755 A1
(43) Pub. Date: Feb. 12, 2026

(54) ALPHAVIRUS T CELL EPITOPES, MEGAPOOLS AND USES THEREOF

(71) Applicant: La Jolla Institute for Immunology, La Jolla, CA (US)

(72) Inventors: Alessandro Sette, La Jolla, CA (US); Daniela Weiskopf, La Jolla, CA (US)

(21) Appl. No.: 19/366,734

(22) Filed: Oct. 23, 2025

Related U.S. Application Data
(63) Continuation-in-part of application No. PCT/US2024/047624, filed on Sep. 20, 2024.
(60) Provisional application No. 63/539,459, filed on Sep. 20, 2023.

(51) Int. Cl. A61K 39/12; A61K 38/20; A61K 38/21; A61K 39/00; A61K 39/385; A61P 31/14; C07K 14/005; C07K 14/74; C12N 7/00; G01N 33/569; G01N 33/68

(52) U.S. Cl. CPC A61K 39/12; A61K 38/204; A61K 38/2066; A61K 38/217; A61K 39/385; A61P 31/14; C07K 14/005; C07K 14/70539; C12N 7/00; G01N 33/56972; G01N 33/56983; G01N 33/6893; A61K 2039/572; A61K 2039/605; C07K 2319/00; C12N 2770/36122; C12N 2770/36134; G01N 2333/181; G01N 2333/70539

(57) ABSTRACT

The present invention includes compositions, including epitope megapools, and methods for detecting the presence of: an Alphavirus or an immune response relevant to an Alphavirus infection including T cells responsive to one or more Alphavirus peptides or proteins comprising, consisting of, or consisting essentially of: one or more amino acid sequences selected from any sequence set forth in Table 1 (SEQ ID NOS: 1 to 150), or a subsequence, portion, homologue, variant or derivative thereof; a fusion protein; a pool of 2 or more peptides; a polynucleotide that encodes one or more peptides or proteins. The invention further provides vaccines, diagnostics, therapies, and kits, comprising such proteins or peptides.

### CROSS-REFERENCE TO RELATED APPLICATIONS

This application is a Continuation-in-Part of and claims priority to Patent Cooperation Treaty filing No. PCT/US2024/047624, filed Sep. 20, 2024, which claims priority to U.S. Provisional Application Ser. No. 63/539,459, filed Sep. 20, 2023, and the entire contents of each of which are incorporated herein by reference.

### STATEMENT OF GOVERNMENT SUPPORT

The inventions described in the present disclosure were made with government support under Contract No. 75N93019C00065, awarded by the National Institutes of Health. The government has certain rights in the invention.

### TECHNICAL FIELD

The present invention relates in general to the field of proteins and peptides that are T cell epitopes and/or antigens for Alphavirus, including epitopes and antigens from Chikungunya virus, and more particularly, to compositions and methods for the prevention, treatment, diagnosis, kits, and uses of such T cell epitopes and antigens, including megapools, for use in detecting and characterizing Chikungunya virus specific responses in infection and following vaccination.

### INCORPORATION-BY-REFERENCE OF MATERIALS FILED ON COMPACT DISC

The application contains a Sequence Listing which has been submitted electronically in .XML format and is hereby incorporated by reference in its entirety. Said .XML copy, created on Oct. 22, 2025, is named “LJII1040CIP.xml” and is 131,401 bytes in size. The sequence listing contained in this .XML file is part of the specification and is hereby incorporated by reference herein in its entirety.

### BACKGROUND

Without limiting the scope of the invention, its background is described in connection with the genus Alphavirus.

A need remains for identifying antigens and T cell epitopes for use in diagnostics, treatments, vaccines, kits, etc., for Alphavirus related diseases and conditions, including Chikungunya. There is additionally a specific need in the art for optimized megapools for use in detecting and characterizing Chikungunya virus specific responses in infection and following vaccination.

### SUMMARY

In one embodiment, the present invention includes a composition comprising: one or more peptides or proteins, comprising, consisting of, or consisting essentially of an amino acid sequence selected from the sequences set forth in Table 1 (SEQ ID NOS: 1 to 150), or a subsequence, portion, homologue, variant or derivative thereof, a fusion protein comprising one or more amino acid sequences selected from any sequence set forth in Table 1 (SEQ ID NOS: 1 to 150); a pool of 2 or more or more peptides comprising, consisting of, or consisting essentially of amino acid sequences selected from any sequence set forth in Table 1 (SEQ ID NOS: 1 to 150); or a polynucleotide that encodes one or more peptides or proteins, comprising, consisting of, or consisting essentially of an amino acid sequence selected from any sequence set forth in Table 1 (SEQ ID NOS: 1 to 150), or a subsequence, portion, homologue, variant or derivative thereof. In one aspect, the one or more peptides or proteins comprises, or wherein the fusion protein comprises 2 or more or more amino acid sequences selected from any sequence set forth in Table 1 (SEQ ID NOS: 1 to 150), or a subsequence, portion, homologue, variant or derivative thereof. In another aspect, the amino acid sequence is selected from an Alphavirus T cell epitope selected from any sequence set forth in Table 1 (SEQ ID NOS: 1 to 150). In another aspect, the composition comprises one or more Chikungunya virus peptides amino acid sequences selected from any sequence set forth in Table 1 (SEQ ID NOS: 1 to 150), or a subsequence, portion, homologue, variant or derivative thereof, a fusion protein comprising one or more amino acid sequences selected from any sequence set forth in Table 1 (SEQ ID NOS: 1 to 150); or a pool of 2 or more peptides selected from any sequence set forth in Table 1 (SEQ ID NOS: 1 to 150). In another aspect, the peptide or protein comprises an Alphavirus T cell epitope. In another aspect, the one or more peptides or proteins comprises an Alphavirus CD8+ or CD4+ T cell epitope. In another aspect, the Alphavirus is Chikungunya virus and the Chikungunya virus T cell epitope is not conserved in another Alphavirus. In another aspect, the Alphavirus is Chikungunya virus and the Chikungunya virus T cell epitope is conserved in another Alphavirus. In another aspect, the one or more peptides or proteins has a length from about 9-15, 15-20, 20-25, 25-30, 30-40, 40-50, 50-75 or 75-100 amino acids. In another aspect, the one or more peptides or proteins elicits, stimulates, induces, promotes, increases or enhances a T cell response to an Alphavirus. In another aspect, the one or more peptides or proteins that elicits, stimulates, induces, promotes, increases or enhances the T cell response to the Alphavirus is an Alphavirus protein or peptide, or a variant, homologue, derivative or subsequence thereof. In another aspect, the composition further comprises formulating the one or more peptides or proteins into an immunogenic formulation with an adjuvant. In another aspect, the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands. In another aspect, the composition further comprises a modulator of immune response. In another aspect, the modulator of immune response is a modulator of the innate immune response. In another aspect, the modulator is Interleukin-6 (IL-6), Interferon-gamma (IFN-γ), Transforming growth factor beta (TGF-β), or Interleukin-10 (IL-10), or an agonist or antagonist thereof.

In another embodiment, the present invention includes a composition comprising monomers or multimers of: peptides or proteins comprising, consisting of, or consisting essentially of: one or more amino acid sequences selected from any sequence set forth in Table 1 (SEQ ID NOS: 1 to 150), concatemers, subsequences, portions, homologues, variants or derivatives thereof, a fusion protein comprising one or more amino acid sequences selected from any sequence set forth in Table 1 (SEQ ID NOS: 1 to 150); or a polynucleotide that encodes one or more peptides or proteins, comprising, consisting of, or consisting essentially of an amino acid sequence selected from any sequence set forth in Table 1 (SEQ ID NOS: 1 to 150), or a subsequence, portion, homologue, variant or derivative thereof.

In another embodiment, the present invention includes a composition comprising one or more peptide-major histocompatibility complex (MHC) monomers or multimers, wherein the peptide-MHC monomer or multimer comprises a peptide comprising, consisting of, or consisting essentially of an amino acid sequence selected from any sequence set forth in Table 1 (SEQ ID NOS: 1 to 150), in a groove of the MHC monomer or multimer.

In another embodiment, the present invention includes a composition comprising: one or more peptides or proteins comprising, consisting of, or consisting essentially of an amino acid sequence selected from any sequence set forth in Table 1 (SEQ ID NOS: 1 to 150), or a subsequence, portion, homologue, variant or derivative thereof, a fusion protein comprising one or more amino acid sequences selected from any sequence set forth in Table 1 (SEQ ID NOS: 1 to 150); a pool of 2 or more peptides selected from any sequence set forth in Table 1 (SEQ ID NOS: 1 to 150); a polynucleotide that encodes one or more peptides or proteins, comprising, consisting of, or consisting essentially of an amino acid sequence selected from any sequence set forth in Table 1 (SEQ ID NOS: 1 to 150), or a subsequence, portion, homologue, variant or derivative thereof. In one aspect, the one or more peptides or proteins comprises, or wherein the fusion protein comprises, 2 or more amino acid sequences selected from any sequence set forth in Table 1 (SEQ ID NOS: 1 to 150), or a subsequence, portion, homologue, variant or derivative thereof. In another aspect, the protein or peptide comprises a Chikungunya virus T cell epitope. In another aspect, the one or more peptides or proteins comprises a Chikungunya virus CD8+ or CD4+ T cell epitope. In another aspect, the Chikungunya virus T cell epitope is not conserved in another Alphavirus. In another aspect, the Chikungunya virus T cell epitope is conserved in another Alphavirus. In another aspect, the one or more peptides or proteins has a length from about 9-15, 15-20, 20-25, 25-30, 30-40, 40-50, 50-75 or 75-100 amino acids. In another aspect, the one or more peptides or proteins elicits, stimulates, induces, promotes, increases or enhances a T cell response to Chikungunya virus. In another aspect, the one or more peptides or proteins that elicits, stimulates, induces, promotes, increases or enhances the T cell response to Chikungunya virus is a Chikungunya virus protein or peptide, or a variant, homologue, derivative or subsequence thereof. In another aspect, the composition further comprises formulating the one or more peptides or proteins into an immunogenic formulation with an adjuvant. In another aspect, the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands. In another aspect, the composition further comprises a modulator of immune response. In another aspect, the modulator of immune response is a modulator of the innate immune response. In another aspect, the modulator is Interleukin-6 (IL-6), Interferon-gamma (IFN-γ), Transforming growth factor beta (TGF-β), or Interleukin-10 (IL-10), or an agonist or antagonist thereof.

In another embodiment, the present invention includes a composition comprising monomers or multimers of: one or more peptides or proteins comprising, consisting of, or consisting essentially of: one or more Chikungunya virus amino acid sequences selected from any sequence set forth in Table 1 (SEQ ID NOS: 1 to 150), concatemers, subsequences, portions, homologues, variants or derivatives thereof, a fusion protein comprising one or more amino acid sequences selected from any sequence set forth in Table 1 (SEQ ID NOS: 1 to 150); or a polynucleotide that encodes one or more peptides or proteins, comprising, consisting of, or consisting essentially of an amino acid sequence selected from any sequence set forth in Table 1 (SEQ ID NOS: 1 to 150), or a subsequence, portion, homologue, variant or derivative thereof.

In another embodiment, the present invention includes a composition comprising one or more peptide-major histocompatibility complex (MHC) monomers or multimers, wherein the peptide-MHC monomer or multimer comprises a peptide comprising, consisting of, or consisting essentially of an amino acid sequence selected from any sequence set forth in Table 1 (SEQ ID NOS: 1 to 150), in a groove of the (MHC) monomer or multimer.

In another embodiment, the present invention includes a method for detecting the presence of: (i) an Alphavirus or (ii) an immune response relevant to Alphavirus infections, vaccines or therapies, including T cells responsive to one or more Alphavirus peptides, comprising: providing one or more proteins or peptides for detection of an amount or a relative amount of, and/or the activity of, and/or the state of antigen-specific T-cells; contacting a biological sample suspected of having Alphavirus-specific T-cells to one or more proteins or peptides for detection; and detecting an amount or a relative amount of, and/or the activity of, and/or the state of antigen-specific T-cells in the biological sample, wherein the one or more proteins or peptides for detection comprise one or more amino acid sequences set forth in Table 1 (SEQ ID NOS: 1 to 150), or comprise a pool of 2 or more or more amino acid sequences set forth in Table 1 (SEQ ID NOS: 1 to 150). In one aspect, detecting the amount or a relative amount of, and/or activity of antigen-specific T-cells comprises one or more steps of identification or detection of the antigen-specific T-cells and measuring the amount of the antigen-specific T-cells. In another aspect, the one or more peptides or proteins comprises 2 or more amino acid sequences selected from Table 1 (SEQ ID NOS: 1 to 150). In another aspect, the detecting the amount or a relative amount of, and/or activity of antigen-specific T-cells comprises indirect detection and/or direct detection. In another aspect, the method of detecting an immune response relevant to the Alphavirus comprises the following steps: providing an MHC monomer or an MHC multimer; contacting a population T-cells to the MHC monomer or MHC multimer; and measuring the number, activity or state of T-cells specific for the MHC monomer or MHC multimer. In one aspect, the MHC monomer or MHC multimer comprises a protein or peptide of the Alphavirus. In another aspect, the protein or peptide comprises a CD8+ or CD4+ T cell epitope. In another aspect, the T cell epitope is not conserved in another Alphavirus. In another aspect, the T cell epitope is conserved in another Alphavirus. In another aspect, the protein or peptide has a length from about 9-15, 15-20, 20-25, 25-30, 30-40, 40-50, 50-75 or 75-100 amino acids. In another aspect, the proteins or peptides comprise 2 or more amino acid sequences selected from any sequence set forth in Table 1 (SEQ ID NOS: 1 to 150), or a subsequence, portion, homologue, variant or derivative thereof. In another aspect, the method further comprises detecting the presence or amount of the one or more peptides in a biological sample, or a response thereto, which is diagnostic of an Alphavirus infection. In another aspect, the detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the method further comprises administering a treatment comprising the composition of one or more proteins, peptides or multimers to the subject from which the biological sample was drawn that increases the amount or relative amount of, and/or activity of the antigen-specific T-cells.

In another embodiment, the present invention includes a method for detecting the presence of: (i) Chikungunya virus or (ii) an immune response relevant to Chikungunya virus infections, vaccines or therapies, including T cells responsive to one or more Chikungunya virus peptides, comprising: providing one or more proteins or peptides for detection of an amount or a relative amount of, and/or the activity of, and/or the state of antigen-specific T-cells; contacting a biological sample suspected of having Chikungunya virus-specific T-cells to one or more proteins or peptides for detection; and detecting an amount or a relative amount of, and/or the activity of, and/or the state of antigen-specific T-cells in the biological sample, wherein the one or more proteins or peptides for detection comprise one or more amino acid sequences set forth in those sequences set forth in Table 1 (SEQ ID NOS: 1 to 150), or comprise a pool of 2 or more amino acid sequences set forth in those sequences set forth in Table 1 (SEQ ID NOS: 1 to 150). In one aspect, detecting the amount or a relative amount of, and/or activity of antigen-specific T-cells comprises one or more steps of identification or detection of the antigen-specific T-cells and measuring the amount of the antigen-specific T-cells. In another aspect, the one or more peptides or proteins comprises 2 or more amino acid sequences selected from any sequence set forth in Table 1 (SEQ ID NOS: 1 to 150). In another aspect, detecting the amount or a relative amount of, and/or activity of antigen-specific T-cells comprises indirect detection and/or direct detection. In another aspect, detecting an immune response relevant to Chikungunya virus comprises the following steps: providing an MHC monomer or an MHC multimer; contacting a population T-cells to the MHC monomer or MHC multimer; and measuring the number, activity or state of T-cells specific for the MHC monomer or MHC multimer. In another aspect, the MHC monomer or MHC multimer comprises a protein or peptide of Chikungunya virus. In another aspect, the protein or peptide comprises a Chikungunya virus CD8+ or CD4+ T cell epitope. In another aspect, the Chikungunya virus T cell epitope is not conserved in another Alphavirus. In another aspect, the Chikungunya virus T cell epitope is conserved in another Alphavirus. In another aspect, the protein or peptide has a length from about 9-15, 15-20, 20-25, 25-30, 30-40, 40-50, 50-75 or 75-100 amino acids. In another aspect, the proteins or peptides comprise 2 or more amino acid sequences selected from any sequence set forth in Table 1 (SEQ ID NOS: 1 to 150), or a subsequence, portion, homologue, variant or derivative thereof. In another aspect, the method further comprises detecting the presence or amount of the one or more peptides in a biological sample, or a response thereto, which is diagnostic of a Chikungunya virus infection. In another aspect, detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the method further comprises administering a treatment comprising the composition of one or more proteins, peptides or multimers to the subject from which the biological sample was drawn that increases the amount or relative amount of, and/or activity of the antigen-specific T-cells.

In another embodiment, the present invention includes a method detecting an Alphavirus infection or exposure in a subject, the method comprising, consisting of, or consisting essentially of: contacting a biological sample from a subject with a composition of composition of one or more proteins, peptides or multimers; and determining if the composition elicits an immune response from the contacted cells, wherein the presence of an immune response indicates that the subject has been exposed to or infected with Alphavirus. In one aspect, the sample comprises T cells. In another aspect, the response comprises inducing, increasing, promoting or stimulating anti-Alphavirus activity of T cells. In another aspect, the T cells are CD8+ or CD4+ T cells. In another aspect, the method comprises determining whether the subject has been infected by or exposed to the Alphavirus more than once by determining if the subject elicits a secondary T cell immune response profile that is different from a primary T cell immune response profile. In another aspect, the method further comprises diagnosing an Alphavirus infection or exposure in a subject, the method comprising contacting a biological sample from a subject with a composition of composition of one or more proteins, peptides or multimers, and determining if the composition elicits a T cell immune response, wherein the T cell immune response identifies that the subject has been infected with or exposed to an Alphavirus. In another aspect, the method is conducted three or more days following the date of suspected infection by or exposure to an Alphavirus.

In another embodiment, the present invention includes a method detecting Chikungunya virus infection or exposure in a subject, the method comprising, consisting of, or consisting essentially of: contacting a biological sample from a subject with a composition of composition of one or more proteins, peptides or multimers; and determining if the composition elicits an immune response from the contacted cells, wherein the presence of an immune response indicates that the subject has been exposed to or infected with Chikungunya virus. In another aspect, the sample comprises T cells. In another aspect, the response comprises inducing, increasing, promoting or stimulating anti-Chikungunya virus activity of T cells. In another aspect, the T cells are CD8+ or CD4+ T cells. In another aspect, the method comprises determining whether the subject has been infected by or exposed to Chikungunya virus more than once by determining if the subject elicits a secondary T cell immune response profile that is different from a primary T cell immune response profile. In another aspect, the method further comprises diagnosing a Chikungunya virus infection or exposure in a subject, the method comprising contacting a biological sample from a subject with a composition of one or more proteins, peptides or multimers; and determining if the composition elicits a T cell immune response, wherein the T cell immune response identifies that the subject has been infected with or exposed to Chikungunya virus. In another aspect, the method is conducted three or more days following the date of suspected infection by or exposure to an Alphavirus.

In another embodiment, the present invention includes a kit for the detection of Alphavirus or an immune response to Alphavirus in a subject comprising, consisting of or consisting essentially of: one or more T cells that specifically detect the presence of: one or more amino acid sequences selected from any sequence set forth in Table 1 (SEQ ID NOS: 1 to 150), or a subsequence, portion, homologue, variant or derivative thereof, or a fusion protein comprising one or more amino acid sequences selected from any sequence set forth in Table 1 (SEQ ID NOS: 1 to 150); or a pool of 2 or more or more peptides selected from the amino acid sequences set forth in Table 1 (SEQ ID NOS: 1 to 150). In one aspect, the one or more amino acid sequences are selected from an Alphavirus T cell epitope set forth in Table 1 (SEQ ID NOS: 1 to 150). In another aspect, the composition comprises: one or more amino acid sequences selected from any sequence set forth in Table 1 (SEQ ID NOS: 1 to 150), or a subsequence, portion, homologue, variant or derivative thereof, a fusion protein comprising one or more amino acid sequences selected from any sequence set forth in Table 1 (SEQ ID NOS: 1 to 150); or a pool of 2 or more peptides selected from the amino acid sequences set forth in those sequences set forth in Table 1 (SEQ ID NOS: 1 to 150). In another aspect, the amino acid sequence comprises an Alphavirus CD8+ or CD4+ T cell epitope. In another aspect, the T cell epitope is not conserved in another Alphavirus. In another aspect, the T cell epitope is conserved in another Alphavirus. In another aspect, the fusion protein has a length from about 9-15, 15-20, 20-25, 25-30, 30-40, 40-50, 50-75 or 75-100 amino acids. In another aspect, the kit includes instruction for a diagnostic method, a process, a composition, a product, a service or component part thereof for the detection of: (i) Alphavirus or (ii) an immune response relevant to Alphavirus infections, vaccines or therapies, including T cells responsive to Alphavirus. In another aspect, the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the kit includes reagents for determining a Human Leukocyte Antigen (HLA) profile of a subject, and selecting peptides that are presented by the HLA profile of the subject for detecting an immune response to Alphavirus.

In another embodiment, the present invention includes a kit for the detection of Chikungunya virus or an immune response to Chikungunya virus in a subject comprising, consisting of or consisting essentially of: one or more T cells that specifically detect the presence of: one or more amino acid sequences selected from any sequence set forth in Table 1 (SEQ ID NOS: 1 to 150), or a subsequence, portion, homologue, variant or derivative thereof, a fusion protein comprising one or more amino acid sequences selected from any sequence set forth in Table 1 (SEQ ID NOS: 1 to 150); or a pool of 2 or more peptides selected from the amino acid sequences set forth in those sequences set forth in Table 1 (SEQ ID NOS: 1 to 150). In another aspect, the amino acid sequence comprises a Chikungunya virus CD8+ or CD4+ T cell epitope. In another aspect, the Chikungunya virus T cell epitope is not conserved in another Alphavirus. In another aspect, the Chikungunya virus T cell epitope is conserved in another Alphavirus. In another aspect, the fusion protein has a length from about 9-15, 15-20, 20-25, 25-30, 30-40, 40-50, 50-75 or 75-100 amino acids. In another aspect, the kit includes instruction for a diagnostic method, a process, a composition, a product, a service or component part thereof for the detection of: (i) Chikungunya virus or (ii) an immune response relevant to Chikungunya virus infections, vaccines or therapies, including T cells responsive to Chikungunya virus. In another aspect, the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the kit includes reagents for determining a Human Leukocyte Antigen (HLA) profile of a subject, and selecting peptides that are presented by the HLA profile of the subject for detecting an immune response to Chikungunya virus.

In another embodiment, the present invention includes a method of stimulating, inducing, promoting, increasing, or enhancing an immune response against an Alphavirus in a subject, comprising: administering a composition of one or more proteins, peptides, multimers or a polynucleotide that expresses the protein, peptide or multimers, in an amount sufficient to stimulate, induce, promote, increase, or enhance an immune response against the Alphavirus in the subject. In another aspect, the immune response provides the subject with protection against an Alphavirus infection or pathology, or one or more physiological conditions, disorders, illnesses, diseases or symptoms caused by or associated with Alphavirus infection or pathology. In another aspect, the immune response is specific to: one or more Chikungunya virus peptides selected from the amino acid sequences set forth in Table 1 (SEQ ID NOS: 1 to 150), or a subsequence, portion, homologue, variant or derivative thereof.

In another embodiment, the present invention includes a method of stimulating, inducing, promoting, increasing, or enhancing an immune response against Chikungunya virus in a subject, comprising: administering a composition of proteins, peptides, multimers or a polynucleotide that expresses the protein, peptide or multimers, in an amount sufficient to stimulate, induce, promote, increase, or enhance an immune response against Chikungunya virus in the subject. In one aspect, the immune response provides the subject with protection against a Chikungunya virus infection or pathology, or one or more physiological conditions, disorders, illnesses, diseases or symptoms caused by or associated with Chikungunya virus infection or pathology. In another aspect, the immune response is specific to: one or more Chikungunya virus peptides selected from the amino acid sequences set forth in those sequences set forth in Table 1 (SEQ ID NOS: 1 to 150), or a subsequence, portion, homologue, variant or derivative thereof.

In another embodiment, the present invention includes a method of stimulating, inducing, promoting, increasing, or enhancing an immune response against Chikungunya virus in a subject, comprising: administering to a subject an amount of a protein or peptide comprising, consisting of or consisting essentially of an amino acid sequence of a Chikungunya virus protein or peptide, or a variant, homologue, derivative or subsequence thereof, wherein the protein or peptide comprises at least two peptides selected from the amino acid sequences set forth in Table 1 (SEQ ID NOS: 1 to 150) or a subsequence, portion, homologue, variant or derivative thereof, in an amount sufficient to prevent, stimulate, induce, promote, increase, immunize against, or enhance an immune response against Chikungunya virus in the subject. In one aspect, the immune response provides the subject with protection against Chikungunya virus infection or pathology, or one or more physiological conditions, disorders, illnesses, diseases or symptoms caused by or associated with Chikungunya virus infection or pathology.

In another embodiment, the present invention includes a method of treating, preventing, or immunizing a subject against Chikungunya virus infection, comprising administering to a subject an amount of a protein or peptide comprising, consisting of, or consisting essentially of an amino acid sequence of an Alphavirus protein or peptide, or a variant, homologue, derivative or subsequence thereof, wherein the protein or peptide comprises at least two amino acid sequences selected from Table 1 (SEQ ID NOS: 1 to 150) or a subsequence, portion, homologue, variant or derivative thereof, in an amount sufficient to treat, prevent, or immunize the subject for Chikungunya virus infection, wherein the protein or peptide comprises or consists of an Alphavirus T cell epitope that elicits, stimulates, induces, promotes, increases, or enhances an anti-Chikungunya virus T cell immune response. In one aspect, the one or more amino acid sequences are selected from any sequence set forth in Table 1 (SEQ ID NOS: 1 to 150), or a subsequence, portion, homologue, variant or derivative thereof, a fusion protein comprising one or more amino acid sequences selected from any sequence set forth in Table 1 (SEQ ID NOS: 1 to 150); or a pool of 2 or more peptides selected from the amino acid sequences set forth in those sequences set forth in Table 1 (SEQ ID NOS: 1 to 150). In one aspect, the anti-Chikungunya virus T cell response is a CD8+, a CD4+ T cell response, or both. In another aspect, the T cell epitope is conserved across two or more clinical isolates of Chikungunya virus or two or more circulating forms of Chikungunya virus. In another aspect, the Chikungunya virus infection is an acute infection. In another aspect, the subject is a mammal or a human. In another aspect, the method reduces Chikungunya viral titer, increases or stimulates Chikungunya viral clearance, reduces or inhibits Chikungunya viral proliferation, reduces or inhibits increases in Chikungunya viral titer or Chikungunya viral proliferation, reduces the amount of a Chikungunya viral protein or the amount of a Chikungunya virus nucleic acid, or reduces or inhibits synthesis of a Chikungunya viral protein or a Chikungunya viral nucleic acid. In another aspect, the method reduces one or more adverse physiological conditions, disorders, illness, diseases, symptoms or complications caused by or associated with Chikungunya virus infection or pathology. In another aspect, the method improves one or more adverse physiological conditions, disorders, illness, diseases, symptoms or complications caused by or associated with Chikungunya virus infection or pathology. In another aspect, the symptom is fever or chills, joint pain, fatigue, muscle or body aches or pains, headache, nausea or vomiting, diarrhea, conjunctivitis or rash. In another aspect, the method reduces or inhibits susceptibility to Chikungunya virus infection or pathology. In another aspect, the protein or peptide, or a subsequence, portion, homologue, variant or derivative thereof, is administered prior to, substantially contemporaneously with or following exposure to or infection of the subject with Chikungunya virus. In another aspect, a plurality of Chikungunya virus T cell epitopes are administered prior to, substantially contemporaneously with or following exposure to or infection of the subject with Chikungunya virus. In another aspect, the protein or peptide, or a subsequence, portion, homologue, variant or derivative thereof is administered within 2-72 hours, 2-48 hours, 4-24 hours, 4-18 hours, or 6-12 hours after a symptom of Chikungunya virus infection or exposure develops. In another aspect, the protein or peptide, or a subsequence, portion, homologue, variant or derivative thereof is administered prior to exposure to or infection of the subject with Chikungunya virus. In another aspect, the method further comprises administering a modulator of immune response prior to, substantially contemporaneously with or following the administration to the subject of an amount of a protein or peptide. In another aspect, the modulator of immune response is a modulator of the innate immune response. In another aspect, the modulator is IL-6, IFN-γ, TGF-β, or IL-10, or an agonist or antagonist thereof.

In another embodiment, the present invention includes a method of treating, preventing, or immunizing a subject against Chikungunya virus infection, comprising administering to a subject the composition of one or more proteins, peptides or multimers in an amount sufficient to treat, prevent, or immunize the subject for Chikungunya virus infection. In one aspect, the Chikungunya virus infection is an acute infection. In another aspect, the method reduces Chikungunya viral titer, increases or stimulates Chikungunya viral clearance, reduces or inhibits Chikungunya viral proliferation, reduces or inhibits increases in Chikungunya viral titer or Chikungunya viral proliferation, reduces the amount of a Chikungunya viral protein or the amount of a Chikungunya viral nucleic acid, or reduces or inhibits synthesis of a Chikungunya viral protein or a Chikungunya viral nucleic acid. In another aspect, the method reduces one or more adverse physiological conditions, disorders, illness, diseases, symptoms or complications caused by or associated with Chikungunya virus infection or pathology. In another aspect, the method improves one or more adverse physiological conditions, disorders, illness, diseases, symptoms or complications caused by or associated with Chikungunya virus infection or pathology. In another aspect, the symptom is fever or chills, joint pain, fatigue, muscle or body aches, headache, nausea or vomiting, diarrhea, conjunctivitis or rash. In another aspect, the method reduces or inhibits susceptibility to Chikungunya virus infection or pathology. In another aspect, the composition is administered prior to, substantially contemporaneously with or following exposure to or infection of the subject with Chikungunya virus. In another aspect, the composition is administered prior to, substantially contemporaneously with or following exposure to or infection of the subject with Chikungunya virus. In another aspect, the composition is administered within 2-72 hours, 2-48 hours, 4-24 hours, 4-18 hours, or 6-12 hours after a symptom of Chikungunya virus infection or exposure develops. In another aspect, the composition is administered prior to exposure to or infection of the subject with Chikungunya virus.

In another embodiment, the present invention includes a peptide or peptides that are immunoprevalent or immunodominant in a virus obtained by a method consisting of, or consisting essentially of: obtaining an amino acid sequence of the virus; determining one or more sets of overlapping peptides spanning one or more virus antigen using unbiased selection; synthesizing one or more pools of viral peptides comprising the one or more sets of overlapping peptides; combining the one or more pools of viral peptides with Class I major histocompatibility proteins (MHC), Class II MHC, or both Class I and Class II MHC to form peptide-MHC complexes; contacting the peptide-MHC complexes with T cells from subjects exposed to the virus; determining which pools triggered cytokine release by the T cells; and deconvoluting from the pool of peptides that elicited cytokine release by the T cells, which peptide or peptides are immunoprevalent or immunodominant in the pool. In one aspect, the virus is an Alphavirus. In another aspect, the Alphavirus is Chikungunya virus. In another aspect, the immunodominant peptides are selected from 1, 2 or more peptides selected from the amino acid sequences set forth in Table 1 (SEQ ID NOS: 1 to 150). In another aspect, the immunodominant peptides are selected from 1, 2 or more peptides selected from the amino acid sequences set forth in those sequences set forth in Table 1 (SEQ ID NOS: 1 to 150).

In another embodiment, the present invention includes a method of selecting an immunoprevalent or immunodominant peptide or protein of a viral comprising, consisting of, or consisting essentially of: obtaining an amino acid sequence of the virus; determining one or more sets of overlapping peptides spanning one or more virus antigen using unbiased selection; synthesizing one or more pools of virus peptides comprising the one or more sets of overlapping peptides; combining the one or more pools of viral peptides with Class I major histocompatibility proteins (MHC), Class II MHC, or both Class I and Class II MHC to form peptide-MHC complexes; contacting the peptide-MHC complexes with T cells from subjects exposed to the virus; determining which pools triggered cytokine release by the T cells; and deconvoluting from the pool of peptides that elicited cytokine release by the T cells, which peptide or peptides are immunoprevalent or immunodominant in the pool. In one aspect, the virus is an Alphavirus. In another aspect, the Alphavirus is Chikungunya virus. In another aspect, the immunodominant peptides are selected from 1, 2 or more peptides selected from the amino acid sequences set forth in Table 1 (SEQ ID NOS: 1 to 150). In another aspect, the immunodominant peptides are selected from 1, 2 or more peptides selected from the amino acid sequences set forth in those sequences set forth in Table 1 (SEQ ID NOS: 1 to 150).

### BRIEF DESCRIPTION OF THE DRAWINGS

For a more complete understanding of the features and advantages of the present disclosure, reference is now made to the detailed description of the disclosure along with the accompanying figures and in which:

FIGS. 1A to 1C shows the experimental workflow for screening CD4⁺ T cell epitopes in CHIKV. (FIG. 1A) Schematic representation of CHIKV proteome comprising four non-structural proteins (nsP1, nsP2, nsP3 and nsP4) and five structural proteins (Capsid or CP, E3, E2 and E1). (FIG. 1B) Workflow of epitope screening. All donors were tested in the AIM assay by stimulation with 10 megapools (MP) corresponding to each CHIKV protein (nsP1, nsP2, nsP3, nsP4, CP, E3, E2, 6K, E1). Positive donors in the AIM assay (OX40+CD137+ or OX40+CD40L+) were tested in the FluoroSpot assay by stimulating with smaller pools of MP, called mesopools, each of which had 9-10 individual peptides. Each mesopool was deconvoluted to determine individual epitopes. (FIG. 1C) An example of the experimental workflow for the responses of one donor to the E1 protein. The first panel shows responses in the AIM assay to all CHIKV MPs. The middle panel shows SFCs per million PBMCs to each mesopool of the E1 protein. The third panel depicts responses to individual peptides in E1-7 mesopool. The dotted line shows the threshold of positivity. The blue highlighted bars depict an example of a positive response from one donor. (SEQ ID NOS: 148-157).

FIGS. 2A to 2G shows the summary of CD4⁺ T cell epitopes identified in 17 individuals with chronic CHIKV disease. (FIG. 2A) Frequency of epitopes detected per CHIKV protein. n refers to the number of total epitopes identified. (FIG. 2B) The magnitude of all positive responses, shown as the total number of IFNγ spot-forming cells per million PBMCs (SFC/10⁶ PBMCs). (FIG. 2C) Percentage of total of average response for all donors plotted as the function of the total number of epitopes. Epitopes were ranked in descending order of average magnitude and percentage of total magnitude was calculated based on cumulative sum of response. Dotted lines represent the number of epitopes that account for 50%, 75% and 90% of total responses. (FIG. 2D) Number of epitopes recognized by each donor. An average of 12 epitopes were identified. (FIG. 2E) Pie chart indicates the frequency of response for each epitope. Parenthesis indicates the number of epitopes recognized by the specified number of donors. (FIG. 2F) The magnitude of positive response to each epitope in the entire CHIKV proteome. The heatmap under the graph show the number of donors who recognize each epitope, ranging from 0-9 donors. The percent shows the percent of overall magnitude of responses elicited by each protein. (FIG. 2G) The lower 95% confidence interval of response frequency of each epitope plotted for the non-structural (top) and structural polyprotein (bottom). The dotted line indicates the threshold of positivity (0.2). The highlighted regions show the epitopes in the regions that reach above threshold of positivity. Data are represented as mean±SD or geomean±geometric SD. (SEQ ID NOS: 84, 85, 86, 87, upper and SEQ ID NOS: 158, 46, 47, 48, 49, lower).

FIGS. 3A and 3B show CHIKV CD4⁺ T cell specific epitope megapool induces a robust response (FIG. 3A) Frequency of antigen-specific CD4⁺ T cells quantified by AIM assay (OX40+CD40L+) after 24-hour stimulation with CHIKV epitope megapools consisting of epitopes from structural (S), non-structural (NS) and combined structural and non-structural (CHIKV S+NS) proteins in 19 chronic CHIKV donors (CHIKV) and seven CHIKV seronegative donors (Uninfected controls: UC). N represents the number of donors. % Positive refers to the percent of donors that are above the LOS (0.04), indicated by the dotted line. (FIG. 3B) Frequency of specific cytokine producing cells (IFNγ, TNFα and IL-2) from the CHIKV-specific CD4⁺ T cells (AIM+ (OX40+CD40L+)) after 24-hour stimulation with the combined structural and non-structural CHIKV epitope megapool (CHIKV S+NS) in 19 chronic CHIKV donors. (FIG. 3C) Frequency of CHIKV-specific AIM+ CD4⁺ T cell memory subsets (OX40+CD40L+) in 19 chronic CHIKV donors post-stimulation with the CHIKV S+NS epitope MP, based on the expression of CCR7 and CD45RA in AIM+ CD4⁺ T cells as: T naive (CCR7⁺ CD45RA⁺), TCM (T central memory; CCR7⁺ CD45RA⁻), TEM (T effector memory; CCR7⁻ CD45RA⁻) and TEMRA (T effector memory re-expressing CD45RA; CCR7⁻ CD45RA⁺). Data are represented as mean±SD or geomean±geometric SD.

FIG. 4A to 4C show the sequence conservation of CHIKV proteome in arthritogenic and encephalitic alphaviruses. (FIG. 4A) Phylogenetic tree indicating the viral sequences used to calculate the percentage of conservation of CHIKV peptides. The tree is divided into arthritogenic (blue) and encephalitic (green) alphaviral sub-groups. (FIG. 4B) Median percent conservation of CHIKV peptides in arthritogenic (blue) and encephalitic (green) alphaviruses. The left y-axis and gray bars refer to the magnitude of response of each CHIKV epitope. The right y-axis, and blue and green lines refer to median percent conservation for each CHIKV peptide. The solid lines separate each CHIKV protein. The median of percent conservation for each CHIKV protein is shown in the table below for arthritogenic (except CHIKV) and encephalitic alphaviruses. (FIG. 4C) Median percent conservation of each CHIKV epitope in arthritogenic (except CHIKV sequences; blue) and encephalitic (green) alphaviruses separated based on non-structural (nsP1, nsP2, nsP3 and nsP4) and structural (CP, E3, E2, 6K and E1) proteins. Each dot refers to a CHIKV epitope. The median percent conservation for each sub-group is shown below. Dark gray lines refer to immunogenic epitopes (recognized by two or more donors). The dotted lines refer to 67% threshold that has previously been shown to define cross-reactive epitopes.

FIGS. 5A to 5C shows representative gating strategy, AIM+ CD4⁺ T cell responses and threshold of positivity for the FluoroSpot assay. (FIG. 5A) Representative gating strategy to define antigen-specific CD3⁺CD4⁺ cells by the AIM assay. Cells shown here were stimulated with nsP1 protein. (FIG. 5B) Antigen-specific CD4⁺ T cells quantified by AIM (left: OX40+CD137+ and right: OX40+CD40L+) after 24-hour stimulation with all CHIKV MPs in 17 CHIKV seropositive donors with chronic CHIKVD. The dotted line represents the limit of sensitivity (LOS; 0.02%). Data are represented as geomean±geometric SD. (FIG. 5C) Graph shows the IFNγ producing SFC/10⁶ PBMCs for six CHIKV-seronegative donors post in vitro stimulation with all CHIKV mesopools. Responses were plotted in descending order of magnitude of response and the top 1% response (red line) was calculated. 99% of the response was lower than 215 SFC/10⁶ PBMCs, which was used as a threshold of positivity for following experiments.

FIGS. 6A and 6B shows representative gating strategy in the AIM/ICS assay and CHIKV-specific antibody responses. (FIG. 6A) Representative gating strategy to define antigen-specific CD4⁺ T cells (OX40+CD40L+) and their functional profile via ICS staining of cytokines (IFNγ, TNFα and IL-2). Cells shown here were stimulated with the combined CHIKV S+NS epitope MP for initial gating and OX40+CD40L+gating, except with PHA for the cytokines. (FIG. 6B) Net CHIKV-specific IgG titers measured in van independent cohort (Chronic CHIKV: n=19; Uninfected controls: n=7). A threshold of 907 (dotted line) was used to determine seropositivity and to confirm CHIKV infection.

FIGS. 7A to 7C show the immunodominant epitopes in CHIKV recognized by CD4⁺ T cells. SFC/10⁶ PBMCs shown for individual chronic (blue line) and recovered (gray line) donors against 15mer peptides overlapping by 14 residues sequentially spanning the immunodominant region of (FIG. 7A) the nsP1 (chronic: n=3, recovered: n=2), (FIG. 7B) E1 (chronic: n=5, recovered: n=3) and (FIG. 7C) CP (chronic: n=4) regions. The black line shows the average SFC/10⁶PBMCs for all donors shown. Blue highlighted regions and bolded peptide sequences indicate region with the highest response and the common amino acid sequence, respectively. The pie charts on the right depict the percentage of HLA alleles predicted to bind to the peptide sequences (i.e. the mapped epitope and negative control peptides on the N- and C-termini) shown under the pie chart. Chi-square value with degree of freedom and p-value is reported for all peptide sequences. Data are represented as mean±SD or geomean±geometric SD.

### DETAILED DESCRIPTION

While the making and using of various embodiments of the present disclosure are discussed in detail below, it should be appreciated that the present disclosure provides many applicable inventive concepts that can be embodied in a wide variety of specific contexts. The specific embodiments discussed herein are merely illustrative of specific ways to make and use the invention and do not delimit the scope of the invention. Numerous variations, changes, and substitutions will now occur to those skilled in the art without departing from the disclosure. It should be understood that various alternatives to the embodiments of the disclosure described herein may be employed in practicing the invention.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. The terminology herein is used to describe specific aspects of the disclosure, but their usage does not delimit the disclosure, except as outlined in the claims. Unless specifically stated or obvious from context, as used herein, the term “or” is understood to be inclusive.

### Definitions

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may, in embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

Proteins and peptides include isolated and purified forms. Proteins and peptides also include those immobilized on a substrate, as well as amino acid sequences, subsequences, portions, homologues, variants, and derivatives immobilized on a substrate.

Proteins and peptides can be included in compositions, for example, a pharmaceutical composition. In particular embodiments, a pharmaceutical composition is suitable for specific or non-specific immunotherapy or is a vaccine composition.

Isolated nucleic acid (including isolated nucleic acid) encoding the proteins and peptides are also provided. Cells expressing a protein or peptide are further provided. Such cells include eukaryotic and prokaryotic cells, such as mammalian, insect, fungal and bacterial cells.

Methods and uses and medicaments of proteins and peptides of the invention are included. Such methods, uses and medicaments include modulating immune activity of a cell against a pathogen, for example, a virus or virion.

The term “peptide mimetic” or “peptidomimetic” refers to protein-like chain designed to mimic a peptide or protein. Peptide mimetics may be generated by modifying an existing peptide or by designing a compound that mimic peptides, including peptoids and β-peptides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution table providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

A “percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “multimer” refers to a complex comprising multiple monomers (e.g., a protein complex) associated by noncovalent bonds. The monomers be substantially identical monomers, or the monomers may be different. In embodiments, the multimer is a dimer, a trimer, a tetramer, or a pentamer.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein or peptide, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e., fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

As used herein, the term “antigen” and the term “epitope” refers to a molecule or substance capable of stimulating an immune response. In one example, epitopes include but are not limited to a polypeptide and a nucleic acid encoding a polypeptide, wherein expression of the nucleic acid into a polypeptide is capable of stimulating an immune response when the polypeptide is processed and presented on a Major Histocompatibility Complex (MHC) molecule. Generally, epitopes include peptides presented on the surface of cells non-covalently bound to the binding groove of Class I or Class II MHC, such that they can interact with T cell receptors and the respective T cell accessory molecules. However, antigens and epitopes also apply when discussing the antigen binding portion of an antibody, wherein the antibody binds to a specific structure of the antigen.

Proteolytic Processing of Antigens. Epitopes that are displayed by MHC on antigen presenting cells are cleavage peptides or products of larger peptide or protein antigen precursors. For MHC I epitopes, protein antigens are often digested by proteasomes resident in the cell. Intracellular proteasomal digestion produces peptide fragments of about 3 to 23 amino acids in length that are then loaded onto the MHC protein. Additional proteolytic activities within the cell, or in the extracellular milieu, can trim and process these fragments further. Processing of MHC Class II epitopes generally occurs via intracellular proteases from the lysosomal/endosomal compartment. The present invention includes, in one embodiment, pre-processed peptides that are attached to the anti-CD40 antibody (or fragment thereof) that directs the peptides against which an enhanced immune response is sought directly to antigen presenting cells.

The present invention includes methods for specifically identifying the epitopes within antigens most likely to lead to the immune response sought for the specific sources of antigen presenting cells and responder T cells.

As used herein, the term “T cell epitope” refers to a specific amino acid that when present in the context of a Major or Minor Histocompatibility Complex provides a reactive site for a T cell receptor. The T-cell epitopes or peptides that stimulate the cellular arm of a subject's immune system are short peptides of about 8-25 amino acids. T-cell epitopes are recognized by T cells from animals that are immune to the antigen of interest. These T-cell epitopes or peptides can be used in assays such as the stimulation of cytokine release or secretion or evaluated by constructing major histocompatibility (MHC) proteins containing or “presenting” the peptide. Such immunogenically active fragments are often identified based on their ability to stimulate lymphocyte proliferation in response to stimulation by various fragments from the antigen of interest.

As used herein, the term “immunological response” refers to an antigen or composition is the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTL” s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of effector and/or suppressor T-cells and/or gamma-delta T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

TABLE 1
Novel Chikungunya virus specific T cell epitopes identified in human populations.

| | Mesopool | Megapool | Result | Sequence | SEQ ID NO: |
|---|---|---|---|---|---|
| 1 CP-1 | CP-1 | CP | Positive | MEFIPTQTFYNRRYQ | 1 |
| 2 CP-1 | CP-1 | CP | Positive | TQTFYNRRYQPRPWT | 2 |
| 3 CP-1 | CP-1 | CP | Positive | NRRYQPRPWTPRPTI | 3 |
| 4 CP-1 | CP-1 | CP | Positive | PRPWTPRPTIQVIRP | 4 |
| 6 CP-1 | CP-1 | CP | Positive | QVIRPRPRPQRQAGQ | 5 |
| 9 CP-1 | CP-1 | CP | Positive | LAQLISAVNKLTMRA | 6 |
| 10 CP-1 | CP-1 | CP | Positive | SAVNKLTMRAVPQQK | 7 |
| 22 CP-3 | CP-3 | CP | Positive | CMKIENDCIFEVKHE | 8 |
| 23 CP-3 | CP-3 | CP | Positive | NDCIFEVKHEGKVTG | 9 |
| 24 CP-3 | CP-3 | CP | Positive | EVKHEGKVTGYACLV | 10 |
| 25 CP-3 | CP-3 | CP | Positive | GKVTGYACLVGDKVM | 11 |
| 26 CP-3 | CP-3 | CP | Positive | YACLVGDKVMKPAHV | 12 |
| 27 CP-3 | CP-3 | CP | Positive | GDKVMKPAHVKGTID | 13 |
| 28 CP-3 | CP-3 | CP | Positive | KPAHVKGTIDNADLA | 14 |
| 31 CP-4 | CP-4 | CP | Positive | KLAFKRSSKYDLECA | 15 |
| 37 CP-4 | CP-4 | CP | Positive | EKPEGYYNWHHGAVQ | 16 |
| 38 CP-4 | CP-4 | CP | Positive | YYNWHHGAVQYSGGR | 17 |
| 41 CP-5 | CP-5 | CP | Positive | FTIPTGAGKPGDSGR | 18 |
| 44 CP-5 | CP-5 | CP | Positive | PIFDNKGRVVAIVLG | 19 |
| 46 CP-5 | CP-5 | CP | Positive | AIVLGGANEGARTAL | 20 |
| 67 E2-1 | E2-1 | E2 | Positive | STKDNFNVYKATRPY | 21 |
| 78 E2-2 | E2-2 | E2 | Positive | SHDWTKLRYMDNHMP | 22 |
| 81 E2-2 | E2-2 | E2 | Positive | ADAERAGLFVRTSAP | 23 |
| 85 E2-2 | E2-2 | E2 | Positive | TMGHFILARCPKGET | 24 |
| 91 E2-3 | E2-3 | E2 | Positive | THPFHHDPPVIGREK | 25 |
| 92 E2-3 | E2-3 | E2 | Positive | HDPPVIGREKFHSRP | 26 |
| 103 E2-4 | E2-4 | E2 | Positive | GNVKITVNGQTVRYK | 27 |
| 108 E2-5 | E2-5 | E2 | Positive | TTTDKVINNCKVDQC | 28 |
| 110 E2-5 | E2-5 | E2 | Positive | KVDQCHAAVTNHKKW | 29 |
| 111 E2-5 | E2-5 | E2 | Positive | HAAVTNHKKWQYNSP | 30 |
| 185 E1-3 | E1-3 | E1 | Positive | ESCKTEFASAYRAHT | 31 |
| 186 E1-3 | E1-3 | E1 | Positive | EFASAYRAHTASASA | 32 |
| 195 E1-4 | E1-4 | E1 | Positive | IVGPMSSAWTPFDNK | 33 |
| 196 E1-4 | E1-4 | E1 | Positive | SSAWTPFDNKIVVYK | 34 |
| 197 E1-4 | E1-4 | E1 | Positive | PFDNKIVVYKGDVYN | 35 |
| 198 E1-4 | E1-4 | E1 | Positive | IVVYKGDVYNMDYPP | 36 |
| 199 E1-4 | E1-4 | E1 | Positive | GDVYNMDYPPFGAGR | 37 |
| 203 E1-5 | E1-5 | E1 | Positive | DIQSRTPESKDVYAN | 38 |
| 206 E1-5 | E1-5 | E1 | Positive | TQLVLQRPAVGTVHV | 39 |
| 207 E1-5 | E1-5 | E1 | Positive | QRPAVGTVHVPYSQA | 40 |
| 208 E1-5 | E1-5 | E1 | Positive | GTVHVPYSQAPSGFK | 41 |
| 210 E1-5 | E1-5 | E1 | Positive | PSGFKYWLKERGASL | 42 |
| 211 E1-5 | E1-5 | E1 | Positive | YWLKERGASLQHTAP | 43 |
| 219 E1-6 | E1-6 | E1 | Positive | IPEAAFTRVVDAPSL | 44 |
| 220 E1-6 | E1-6 | E1 | Positive | FTRVVDAPSLTDMSC | 45 |
| 225 E1-7 | E1-7 | E1 | Positive | FGGVAIIKYAASKKG | 46 |
| 226 E1-7 | E1-7 | E1 | Positive | IIKYAASKKGKCAVH | 47 |
| 227 E1-7 | E1-7 | E1 | Positive | ASKKGKCAVHSMTNA | 48 |
| 228 E1-7 | E1-7 | E1 | Positive | KCAVHSMTNAVTIRE | 49 |
| 229 E1-7 | E1-7 | E1 | Positive | SMTNAVTIREAEIEV | 50 |
| 232 E1-7 | E1-7 | E1 | Positive | EGNSQLQISFSTALA | 51 |
| 235 E1-8 | E1-8 | E1 | Positive | SAEFRVQVCSTQVHC | 52 |
| 239 E1-8 | E1-8 | E1 | Positive | PPKDHIVNYPASHTT | 53 |
| 240 E1-8 | E1-8 | E1 | Positive | IVNYPASHTTLGVQD | 54 |
| 243 E1-9 | E1-9 | E1 | Positive | ISATAMSWVQKITGG | 55 |
| 244 E1-9 | E1-9 | E1 | Positive | MSWVQKITGGVGLVV | 56 |
| 249 CP-6 | CP-6 | CP | Positive | NRRYQPRPWTPRSTI | 57 |
| 253 CP-6 | CP-6 | CP | Positive | RPRPQRKAGQLAQLI | 58 |
| 255 CP-6 | CP-6 | CP | Positive | LAQLISAVNKLTMRV | 59 |
| 256 CP-6 | CP-6 | CP | Positive | SAVNKLTMRVVPQQK | 60 |
| 265 CP-7 | CP-7 | CP | Positive | KPVQKKKKPGRRERM | 61 |
| 273 E3 | E3 | E3 | Positive | DNVMSPGYYQLLQAS | 62 |
| 290 E2-10 | E2-10 | E2 | Positive | MSQQSGNVKITVNSQ | 63 |
| 291 E2-10 | E2-10 | E2 | Positive | GNVKITVNSQTVRYK | 64 |
| 296 E2-11 | E2-11 | E2 | Positive | ITTDKVINNCKIDQC | 65 |
| 298 E2-11 | E2-11 | E2 | Positive | KIDQCHAAVTNHKKW | 66 |
| 305 E2-11 | E2-11 | E2 | Positive | VTHKKEIRLTVPTEG | 67 |
| 306 E2-12 | E2-12 | E2 | Positive | EIRLTVPTEGLEVTW | 68 |
| 316 E2-12 | E2-12 | E2 | Positive | LLSLLCCIRTAKAAT | 69 |
| 340 E1-10 | E1-10 | E1 | Positive | TQLVLQRPSAGTVHV | 70 |
| 343 E1-11 | E1-11 | E1 | Positive | ATNPVRAMNCAVGNM | 71 |
| 345 E1-11 | E1-11 | E1 | Positive | AVGNMPISIDIPDAA | 72 |
| 346 E1-11 | E1-11 | E1 | Positive | PISIDIPDAAFTRVV | 73 |
| 365 nsP1-2 | nsP1-2 | nsP1 | Positive | DPDSTILDIGSAPAR | 74 |
| 366 nsP1-2 | nsP1-2 | nsP1 | Positive | ILDIGSAPARRMMSD | 75 |
| 367 nsP1-2 | nsP1-2 | nsP1 | Positive | SAPARRMMSDRKYHC | 76 |
| 368 nsP1-2 | nsP1-2 | nsP1 | Positive | RMMSDRKYHCVCPMR | 77 |
| 372 nsP1-2 | nsP1-2 | nsP1 | Positive | ERLANYARKLASAAG | 78 |
| 373 nsP1-2 | nsP1-2 | nsP1 | Positive | YARKLASAAGKVLDR | 79 |
| 388 nsP1-4 | nsP1-4 | nsP1 | Positive | QVAYWVGFDTTPFMY | 80 |
| 389 nsP1-4 | nsP1-4 | nsP1 | Positive | VGFDTTPFMYNAMAG | 81 |
| 390 nsP1-4 | nsP1-4 | nsP1 | Positive | TPFMYNAMAGAYPSY | 82 |
| 392 nsP1-4 | nsP1-4 | nsP1 | Positive | AYPSYSTNWADEQVL | 83 |
| 397 nsP1-5 | nsP1-5 | nsP1 | Positive | DLTEGRRGKLSIMRG | 84 |
| 398 nsP1-5 | nsP1-5 | nsP1 | Positive | RRGKLSIMRGKKLKP | 85 |
| 399 nsP1-5 | nsP1-5 | nsP1 | Positive | SIMRGKKLKPCDRVL | 86 |
| 400 nsP1-5 | nsP1-5 | nsP1 | Positive | KKLKPCDRVLFSVGS | 87 |
| 406 nsP1-6 | nsP1-6 | nsP1 | Positive | PSVFHLKGKLSFTCR | 88 |
| 407 nsP1-6 | nsP1-6 | nsP1 | Positive | LKGKLSFTCRCDTVV | 89 |
| 409 nsP1-6 | nsP1-6 | nsP1 | Positive | CDTVVSCEGYVVKRI | 90 |
| 412 nsP1-6 | nsP1-6 | nsP1 | Positive | TMSPGLYGKTTGYAV | 91 |
| 464 | nsP2_1-1 | nsP2_1 | Positive | PTDHVVGEYLVLSPQ | 92 |
| nsP2_1-1 | | | | | |
| 492 | nsP2_1-4 | nsP2_1 | Positive | LTNPPYHEFAYEGLK | 93 |
| nsP2_1-4 | | | | | |
| 493 | nsP2_1-4 | nsP2_1 | Positive | YHEFAYEGLKIRPAC | 94 |
| nsP2_1-4 | | | | | |
| 576 | nsP2_1- | nsP2_1 | Positive | KGKWNINKQICVTTR | 95 |
| nsP2_1-12 | 12 | | | | |
| 585 | nsP2_2-1 | nsP2_2 | Positive | GERMEWLVNKINGHH | 96 |
| nsP2_2-1 | | | | | |
| 586 | nsP2_2-1 | nsP2_2 | Positive | WLVNKINGHHVLLVS | 97 |
| nsP2_2-1 | | | | | |
| 596 | nsP2_2-2 | nsP2_2 | Positive | LGRYDLVVINIHTPF | 98 |
| nsP2_2-2 | | | | | |
| 608 | nsP2_2-4 | nsP2_2 | Positive | RVICVLGRKFRSSRA | 99 |
| nsP2_2-4 | | | | | |
| 621 nsP3-1 | nsP3-1 | nsP3 | Positive | SYRVKRMDIAKNDEE | 100 |
| 624 nsP3-1 | nsP3-1 | nsP3 | Positive | CVVNAANPRGLPGDG | 101 |
| 627 nsP3-1 | nsP3-1 | nsP3 | Positive | VCKAVYKKWPESFKN | 102 |
| 629 nsP3-1 | nsP3-1 | nsP3 | Positive | ESFKNSATPVGTAKT | 103 |
| 633 nsP3-2 | nsP3-2 | nsP3 | Positive | YPVIHAVGPNFSNYS | 104 |
| 638 nsP3-2 | nsP3-2 | nsP3 | Positive | AYREVAKEVTRLGVN | 105 |
| 639 nsP3-2 | nsP3-2 | nsP3 | Positive | AKEVTRLGVNSVAIP | 106 |
| 650 nsP3-4 | nsP3-4 | nsP3 | Positive | WEKKISEAIQMRTQV | 107 |
| 665 nsP3-5 | nsP3-5 | nsP3 | Positive | QTEANEQVCLYALGE | 108 |
| 669 nsP3-5 | nsP3-5 | nsP3 | Positive | RQKCPVDDADASSPP | 109 |
| 679 nsP3-6 | nsP3-6 | nsP3 | Positive | PKYKIEGVQKVKCSK | 110 |
| 680 nsP3-7 | nsP3-7 | nsP3 | Positive | EGVQKVKCSKVMLFD | 111 |
| 681 nsP3-7 | nsP3-7 | nsP3 | Positive | VKCSKVMLFDHNVPS | 112 |
| 682 nsP3-7 | nsP3-7 | nsP3 | Positive | VMLFDHNVPSRVSPR | 113 |
| 683 nsP3-7 | nsP3-7 | nsP3 | Positive | HNVPSRVSPREYRSS | 114 |
| 698 nsP3-8 | nsP3-8 | nsP3 | Positive | AVSDWVMSTVPVAPP | 115 |
| 874 | nsP2_2-5 | nsP2_2 | Positive | PRGAIKVTAQLTDHV | 116 |
| nsP2_2-5 | | | | | |
| 875 | nsP2_2-5 | nsP2_2 | Positive | VTAQLTDHVVGEYL | 117 |
| nsP2_2-5 | | | | | |
| 876 | nsP2_2-5 | nsP2_2 | Positive | LTDHVVGEYLVLSPQ | 118 |
| nsP2_2-5 | | | | | |
| 928 nsP3-13 | nsP3-13 | nsP3 | Positive | QESVREVSMTTSLTH | 119 |
| 948 nsP3-16 | nsP3-16 | nsP3 | Positive | HPPISFGAPSETFPI | 120 |
| 952 nsP3-16 | nsP3-16 | nsP3 | Positive | NDGEIESLSSELLTF | 121 |
| | | nsP2_2 | Positive | PRGAIKVTAQPTDHV | 122 |
| | | nsP2_2 | Positive | KVTAQPTDHVVGEYL | 123 |
| | | nsP3 | Positive | FSNYSESEGDRELAA | 124 |
| | | nsP3 | Positive | HPPISFGASSETFPI | 125 |
| | | nsP3 | Positive | NEGEIESLSSELLTF | 126 |
| | | CP | Positive | RPRPQRQAGQLAQLI | 127 |
| | | CP | Positive | KPAQKKKKPGRRERM | 128 |
| | | E3 | Positive | DNVMRPGYYQLLQAS | 129 |
| | | E2 | Positive | MSQQSGNVKITVNGQ | 130 |
| | | E2 | Positive | VMHKKEVVLTVPTEG | 131 |
| | | E2 | Positive | EVVLTVPTEGLEVTW | 132 |
| | | E2 | Positive | LLSLICCIRTAKAAT | 133 |
| | | 6K | Positive | EQQPLFWLQALIPLA | 134 |
| | | E1 | Positive | ATNPVRAVNCAVGNM | 135 |
| | | E1 | Positive | AVGNMPISIDIPEAA | 136 |
| | | E1 | Positive | PISIDIPEAAFTRVV | 137 |
| | | nsP2_2 | Positive | KVTAQLTDHVVGEYL | 138 |
| | | nsP3 | Positive | FSNYTESEGDRELAA | 139 |
| | | 6K | Positive | EQQPLFWMQALIPLA | 140 |
| | | E2 | Positive | FNVYKATRPYLAHCP | 141 |
| | | E1 | Positive | SPYVKCCGTAECKDK | 142 |
| | | nsP3 | Positive | QSLNHLFTAMDSTDA | 143 |
| | | E2 | Positive | ILYYYELYPTMTVVV | 144 |
| | | 6K | Positive | ALIVLCNCLRLLPCC | 145 |
| | | E2 | Positive | CARRRCITPYELTPG | 146 |
| | | nsP2_1 | Positive | FFNMMQMKVNYNHNI | 147 |
| | | | | KLSIMRGKKL | 148 |
| | | | | IKYAASKKG | 149 |
| | | | | LISAVNKLTMR | 150 |

The present invention relates in general to the field of proteins and peptides that are T cell epitopes and/or antigens for Alphavirus, including epitopes and antigens from Chikungunya virus, and more particularly, to compositions and methods for the prevention, treatment, diagnosis, kits, and uses of such T cell epitopes and antigens, including megapools, for use in detecting and characterizing Chikungunya virus specific responses in infection and following vaccination.

In another embodiment, the present invention includes a polynucleotide that expresses one or more peptides or proteins, comprising, consisting of, or consisting essentially of an amino acid sequence selected from any sequence set forth in Table 1 (SEQ ID NOS: 1 to 150), or a subsequence, portion, homologue, variant or derivative thereof, a fusion protein comprising one or more amino acid sequences selected from any sequence set forth in Table 1 (SEQ ID NOS: 1 to 150); or a pool of 2 or more or more peptides comprising, consisting of, or consisting essentially of amino acid sequences selected from any sequence set forth in Table 1 (SEQ ID NOS: 1 to 150). In one aspect, the vector comprises the polynucleotide of claim that expresses one or more peptides or proteins, comprising, consisting of, or consisting essentially of an amino acid sequence selected from any sequence set forth in Table 1 (SEQ ID NOS: 1 to 150), or a subsequence, portion, homologue, variant or derivative thereof, a fusion protein comprising one or more amino acid sequences selected from any sequence set forth in Table 1 (SEQ ID NOS: 1 to 150); or a pool of 2 or more or more peptides comprising, consisting of, or consisting essentially of amino acid sequences selected from any sequence set forth in Table 1 (SEQ ID NOS: 1 to 150), a viral vector, or a host cell the comprises the same.

In another embodiment, the present invention includes a polynucleotide that expresses one or more peptides or proteins comprising, consisting of, or consisting essentially of an amino acid sequence selected from any sequence set forth in Table 1 (SEQ ID NOS: 1 to 150), or a subsequence, portion, homologue, variant or derivative thereof, a fusion protein comprising one or more amino acid sequences selected from any sequence set forth in Table 1 (SEQ ID NOS: 1 to 150); or a pool of 2 or more peptides selected from any sequence set forth in Table 1 (SEQ ID NOS: 1 to 150). In one aspect, the vector comprises the polynucleotide of claim that expresses one or more peptides or proteins comprising, consisting of, or consisting essentially of an amino acid sequence selected from any sequence set forth in Table 1 (SEQ ID NOS: 1 to 150), or a subsequence, portion, homologue, variant or derivative thereof, a fusion protein comprising one or more amino acid sequences selected from any sequence set forth in Table 1 (SEQ ID NOS: 1 to 150); or a pool of 2 or more peptides selected from any sequence set forth in Table 1 (SEQ ID NOS: 1 to 150), a viral vector, or a host cell that comprises the same.

As used herein, the term “Major Histocompatibility Complex” (MHC) is a generic designation meant to encompass the histocompatibility antigen systems described in different species including the human leucocyte antigens (HLA). Typically, MHC Class I or Class II multimers are well known in the art and include but are not limited to dimers, tetramers, pentamers, hexamers, heptamers and octamers.

As used herein, the term “MHC/peptide multimer” refers to a stable multimeric complex composed of MHC protein(s) subunits loaded with a peptide of the present invention. For example, an MHC/peptide multimer (also called herein MHC/peptide complex) include, but are not limited to, an MHC/peptide dimer, trimer, tetramer, pentamer, dextramer, or higher/other valency multimer. Several examples of dextramers are those of IMMUDEX®. In humans there are three major different genetic loci that encode MHC class I molecules (the MHC molecules of the human are also designated human leukocyte antigens (HLA)): HLA-A, HLA-B, HLA-C, e.g., HLA-A*01, HLA-A*02, and HLA-A*11 are examples of different MHC class I alleles that can be expressed from these loci. Non-classical human MHC class I molecules such as HLA-E (homolog of mice Qa-1b) and MICA/B molecules are also encompassed by the present invention. In some embodiments, the MHC/peptide multimer is an HLA/peptide multimer selected from the group consisting of HLA-A/peptide multimer, HLA-B/peptide multimer, HLA-C/peptide multimer, HLA-E/peptide multimer, MICA/peptide multimer and MICB/peptide multimer.

In humans there are three major different genetic loci that encode MHC class II molecules: HLA-DR, HLA-DP, and HLA-DQ, each formed of two polypeptides, alpha and beta chains (A and B genes). For example, HLA-DQA1*01, HLA-DRB1*01, and HLA-DRB1*03 are different MHC class II alleles that can be expressed from these loci. It should be further noted that non-classical human MHC class II molecules such as HLA-DM and HL-DOA (homolog in mice is H2-DM and H2-O) are also encompassed by the present invention. In some embodiments, the MHC/peptide multimer is an HLA/peptide multimer selected from the group consisting of HLA-DP/peptide multimer, HLA-DQ/peptide multimer, HLA-DR/peptide multimer, HLA-DM/peptide multimer and HLA-DO/peptide multimer.

An MHC/peptide multimer may be a multimer where the heavy chain of the MHC is biotinylated, which allows combination as a tetramer with streptavidin. MHC-peptide tetramers have increased avidity for the appropriate T cell receptor (TCR) on T lymphocytes. The multimers can also be attached to paramagnetic particles or magnetic beads to facilitate removal of non-specifically bound reporter and cell sorting. Multimer staining does not kill the labelled cells, thus, cell integrity is maintained for further analysis. In some embodiments, the MHC/peptide multimer of the present invention is particularly suitable for isolating and/or identifying a population of CD8+ T cells having specificity for the peptide of the present invention (in a flow cytometry assay).

The peptides or MHC class I or class II multimer as described herein is particularly suitable for detecting T cells specific for one or more peptides of the present invention. The peptide(s) and/or the MHC/multimer complex of the present invention is particularly suitable for diagnosing Alphavirus infection in a subject. For example, the method comprises obtaining a blood or PBMC sample obtained from the subject with an amount of a least peptide of the present invention and detecting at least one T cell displaying a specificity for the peptide. Another diagnostic method of the present invention involves the use of a peptide of the present invention that is loaded on multimers as described above, so that the isolated CD8+ or CD4+ T cells from the subject are brought into contact with the multimers, at which the binding, activation and/or expansion of the T cells is measured. For example, following the binding to antigen presenting cells, e.g., those having the MHC class I or class II multimer, the number of CD8+ and/or CD4+ cells binding specifically to the HLA-peptide multimer may be quantified by measuring the secretion of lymphokines/cytokines, division of the T cells, or standard flow cytometry methods, such as, for example, using fluorescence activated cell sorting (FACS). The multimers can also be attached to paramagnetic ferrous or magnetic beads to facilitate removal of non-specifically bound reporter and cell sorting. The MHC class I or class II peptide multimers as described herein can also be used as therapeutic agents. The peptide and/or the MHC class I or class II peptide multimers of the present invention are suitable for treating or preventing an Alphavirus infection in a subject. The MHC Class I or Class II multimers can be administered in soluble form or loaded on nanoparticles.

As used herein, the term an “immunogenic composition” and “vaccine” refer to a composition that comprises an antigenic molecule where administration of the composition to a subject or patient results in the development in the subject of a humoral and/or a cellular immune response to the antigenic molecule of interest. “Vaccine” refers to a composition that can provide active acquired immunity to and/or therapeutic effect (e.g., treatment) of a particular disease or a pathogen. A vaccine typically contains one or more agents that can induce an immune response in a subject against a pathogen or disease, i.e., a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g., preventing or ameliorating the effects of a future infection by any natural or pathogen) or therapeutic (e.g., reducing symptoms or aberrant conditions associated with infection). The administration of vaccines is referred to vaccination.

In some examples, a vaccine composition can provide nucleic acid, e.g., mRNA that encodes antigenic molecules (e.g., peptides) to a subject. The nucleic acid that is delivered via the vaccine composition in the subject can be expressed into antigenic molecules and allow the subject to acquire immunity against the antigenic molecules. In the context of the vaccination against infectious disease, the vaccine composition can provide mRNA encoding antigenic molecules that are associated with a certain pathogen, e.g., one or more peptides that are known to be expressed in the pathogen (e.g., pathogenic virus).

### EXAMPLE 1

The present invention provides peptides and the nucleic acid molecules that encode the peptides, specifically polynucleotides, primary constructs and/or mRNA that encode one or more polynucleotides that express one or more peptides or proteins, comprising, consisting of, or consisting essentially of an amino acid sequence selected from any sequence set forth in Table 1, or a subsequence, portion, homologue, variant or derivative thereof for use in immune modulation. The term “nucleic acid” refers to any compound and/or substance that comprise a polymer of nucleotides, referred to herein as polynucleotides. Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs), including diastereomers of LNAs, functionalized LNAs, or hybrids thereof. The one or more of the peptides can be included in a composition, that can include 1, 2, 3, 4, 5, 6, 7, 8, 9, 10, 11, 12, 14, 15, 16, 17, 18, 19, 20, 25, 30, 40, 50, 60, 70, 75, 80, 90, 100, 110, 120, 130, 140, 147, or more of the peptides disclosed herein.

Table 1: Novel Chikungunya virus specific T cell epitopes identified in human populations.

One method of immune modulation of the present invention includes direct or indirect gene transfer, i.e., local application of a preparation containing the one or more polynucleotides (DNA, RNA, mRNA, etc.) that expresses the one or more peptides or proteins, comprising, consisting of, or consisting essentially of an amino acid sequence selected from any sequence set forth in Table 1 (SEQ ID NOS: 1 to 150), or a subsequence, portion, homologue, variant or derivative thereof. A variety of well-known vectors can be used to deliver to cells the one or more polynucleotides or the peptides or proteins expressed by the polynucleotides, including but not limited to adenobacterial vectors and adeno-associated vectors. In addition, naked DNA, liposome delivery methods, or other novel vectors developed to deliver the polynucleotides to cells can also be beneficial. Any of a variety of promoters can be used to drive peptide or protein expression, including but not limited to endogenous promoters, constitutive promoters (e.g., cytomegalobacteria, adenobacteria, or SV40), inducible promoters (e.g., a cytokine promoter such as the interleukin-1, tumor necrosis factor-alpha, or interleukin-6 promoter), and tissue specific promoters to express the immunogenic peptides or proteins of the present invention.

The immunization may include adenobacteria, adeno-associated bacteria, herpes bacteria, vaccinia bacteria, retrobacteriaes, or other bacterial vectors with the appropriate tropism for cells likely to present the antigenic peptide(s) or protein(s) may be used as a gene transfer delivery system for a therapeutic peptide(s) or protein(s), comprising, consisting of, or consisting essentially of an amino acid sequence selected from any sequence set forth in Table 1 (SEQ ID NOS: 1 to 150), or a subsequence, portion, homologue, variant or derivative thereof, gene expression construct. Bacterial vectors which do not require that the target cell be actively dividing, such as adenobacterial and adeno-associated vectors, are particularly useful when the cells are accumulating, but not proliferative. Numerous vectors useful for this purpose are generally known (Miller, Human Gene Therapy 15-14, 1990; Friedman, Science 244:1275-1281, 1989; Eglitis and Anderson, BioTechniques 6:608-614, 1988; Tolstoshev and Anderson, Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; and Miller and Rosman, Bio Techniques 7:980-990, 1989; Le Gal La Salle et al., Science 259:988-990, 1993; and Johnson, Chest 107:77S-83S, 1995). Retrobacterial vectors are particularly well developed and have been used in clinical settings (Rosenberg et al., N. Engl. J. Med 323:370, 1990; Anderson et al., U.S. Pat. No. 5,399,346).

The immunization may also include inserting the one or more polynucleotides (DNA, RNA, mRNA, etc.) that express the one or more peptides or proteins, comprising, consisting of, or consisting essentially of an amino acid sequence selected from any sequence set forth in Table 1 (SEQ ID NOS: 1 to 150), or a subsequence, portion, homologue, variant or derivative thereof into the viral vector, along with another gene which encodes the ligand for a receptor on a specific target cell, for example, such that the vector is now target specific. Bacterial vectors can be made target specific by attaching, for example, a sugar, a glycolipid, or a protein. Targeting can also be accomplished by using an antibody to target the bacterial vector. Those of skill in the art will know of, or can readily ascertain without undue experimentation, specific polynucleotide sequences which can be inserted into the bacterial genome or attached to a bacterial envelope to allow target specific delivery of the bacterial vector containing the gene.

Since recombinant bacteriaes are defective, they require assistance in order to produce infectious vector particles. This assistance can be provided, for example, by using helper cell lines that contain plasmids encoding all of the structural genes of the bacteria under the control of regulatory sequences within the bacterial genome. These plasmids are missing a nucleotide sequence which enables the packaging mechanism to recognize a polynucleotide transcript for encapsidation. These cell lines produce empty virions, since no genome is packaged. If a bacterial vector is introduced into such cells in which the packaging signal is intact, but the structural genes are replaced by other genes of interest, the vector can be packaged and vector virion produced.

Bacterial or non-bacterial approaches may also be employed for the introduction of one or more therapeutic polynucleotides that express the one or more peptides or proteins, comprising, consisting of, or consisting essentially of an amino acid sequence selected from any sequence set forth in Table 1 (SEQ ID NOS: 1 to 150), or a subsequence, portion, homologue, variant or derivative thereof, into polynucleotide-encoding polynucleotide into antigen presenting cells. The polynucleotides may be DNA, RNA, mRNA that directly encode the one or more peptides or proteins of the present invention, or may be introduced as part of an expression vector.

Another example of an immunization includes colloidal dispersion systems that include macromolecule complexes, nanocapsules, microspheres, beads, and lipid-based systems including oil-in-water emulsions, micelles, mixed micelles, and liposomes and the one or more polynucleotides that express the one or more peptides or proteins, comprising, consisting of, or consisting essentially of an amino acid sequence selected from any sequence set forth in Table 1 (SEQ ID NOS: 1 to 150), or a subsequence, portion, homologue, variant or derivative thereof. One non-limiting example of a colloidal system for use with the present invention is a liposome. Liposomes are artificial membrane vesicles which are useful as delivery vehicles in vitro and in vivo. It has been shown that large unilamellar vesicles (LUV), which range in size from 0.2-4.0 micrometers that can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77, 1981). In addition to mammalian cells, liposomes have been used for delivery of polynucleotides in plant, yeast and bacterial cells. In order for a liposome to be an efficient gene transfer vehicle, the following characteristics should be present: (Zakut and Givol, supra) encapsulation of the genes of interest at high efficiency while not compromising their biological activity; (Fearnhead, et al., supra) preferential and substantial binding to a target cell in comparison to non-target cells; (Korsmeyer, S. J., supra) delivery of the aqueous contents of the vesicle to the target cell cytoplasm at high efficiency; and (Kinoshita, et al., supra) accurate and effective expression of genetic information (Mannino, et al., Bio Techniques, 6:682, 1988).

The composition for immunizing the subject or patient may, in certain embodiments comprise a combination of phospholipid, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticuloendothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization, specifically, cells that can become infected with an Alphavirus or interact with the proteins, peptides, and/or gene products of an Alphavirus, e.g., immune cells.

For any of the above approaches, the immune modulating polynucleotide construct, composition, or formulation is preferably applied to a site that will enhance the immune response. For example, the immunization may be intramuscular, intraperitoneal, enteral, parenteral, intranasal, intrapulmonary, or subcutaneous. In the gene delivery constructs of the instant invention, polynucleotide expression is directed from any suitable promoter (e.g., the human cytomegalobacteria, simian bacteria 40, actin or adenobacteria constitutive promoters; or the cytokine or metalloprotease promoters for activated synoviocyte specific expression).

In one example of the immune modifying peptide(s) or protein(s) include polynucleotides, constructs and/or mRNAs that express the one or more polynucleotides that express the one or more peptides or proteins, comprising, consisting of, or consisting essentially of an amino acid sequence selected from any sequence set forth in Table 1 (SEQ ID NOS: 1 to 150), or a subsequence, portion, homologue, variant or derivative thereof, that are designed to improve one or more of the stability and/or clearance in tissues, uptake and/or kinetics, cellular access by the peptide(s) or protein(s), translational, mRNA half-life, translation efficiency, immune evasion, protein production capacity, accessibility to circulation, peptide(s) or protein(s) half-life and/or presentation in the context of MHC on antigen presenting cells.

The present invention contemplates immunization for use in both active and passive immunization embodiments. Immunogenic compositions, proposed to be suitable for use as a vaccine, may be prepared most readily directly from immunogenic peptides, proteins, monomers, multimers and/or peptide-MHC complexes prepared in a manner disclosed herein. The antigenic material is generally processed to remove undesired contaminants, such as, small molecular weight molecules, incomplete proteins, or when manufactured in plant cells, plant components such as cell walls, plant proteins, and the like. Often, these immunizations are lyophilized for ease of transport and/or to increase shelf-life and can then be more readily dissolved in a desired vehicle, such as saline.

The preparation of immunizations (also referred to as vaccines) that contain the immunogenic proteins of the present invention as active ingredients is generally well understood in the art, as exemplified by U.S. Pat. Nos. 4,608,251; 4,601,903; 4,599,231; 4,599,230; 4,596,792; and 4,578,770, all incorporated herein by reference. Typically, such immunizations are prepared as injectable. The immunizations can be a liquid solution or suspension but may also be provided in a solid form suitable for solution in, or suspension in, liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, buffers, or the like and combinations thereof. In addition, if desired, the immunization may contain minor amounts of auxiliary substances such as wetting or emulsifying agents, pH buffering agents, or adjuvants which enhance the effectiveness of the vaccines.

The immunization is/are administered in a manner compatible with the dosage formulation, and in such amount as will be therapeutically effective and immunogenic. The quantity to be administered depends on the subject to be treated, including, e.g., the capacity of the individual's immune system to synthesize antibodies, and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable but are typified by an initial administration followed by subsequent inoculations or other administrations.

The manner of application of the immunization may be varied widely. Any of the conventional methods for administration of a vaccine are applicable. These are believed to also include oral application on a solid physiologically acceptable base or in a physiologically acceptable dispersion, parenterally, by injection or the like. The dosage of the vaccine will depend on the route of administration and will vary according to the size of the host.

Various methods of achieving adjuvant effect for the vaccine includes use of agents such as aluminum hydroxide or phosphate (alum), commonly used as 0.05 to 0.1 percent solution in phosphate buffered saline, admixture with synthetic polymers of sugars (Carbopol) used as 0.25 percent solution, aggregation of the protein in the vaccine by heat treatment with temperatures ranging between 700 to 101° C. for 30 second to 2-minute periods respectively. Aggregation by reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed.

In many instances, it will be desirable to have multiple administrations of the vaccine, usually not exceeding six to ten immunizations, more usually not exceeding four immunizations and preferably one or more, usually at least about three immunizations. The immunizations will normally be at from two to twelve-week intervals, more usually from three to five-week intervals. Periodic boosters at intervals of 1-5 years, usually three years, will be desirable to maintain protective levels of the antibodies. The course of the immunization may be followed by assays for antibodies for the supernatant antigens. The assays may be performed by labeling with conventional labels, such as radionuclides, enzymes, fluorescent agents, and the like. These techniques are well known and may be found in a wide variety of patents, such as Hudson and Cranage, Vaccine Protocols, 2003 Humana Press, relevant portions incorporated herein by reference.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000, and updates thereto; Martindale, The Extra Pharmacopoeia, Thirty-Second Edition (The Pharmaceutical Press, London, 1999); all of which are incorporated by reference, and the like, relevant portions incorporated herein by reference.

Many suitable expression systems are commercially available, including, for example, the following: baculobacteria expression (Reilly, P. R., et al., BACULOBACTERIA EXPRESSION VECTORS: A LABORATORY MANUAL (1992); Beames, et al., Biotechniques 11:378 (1991); Pharmingen; Clontech, Palo Alto, Calif)), vaccinia expression systems (Earl, P. L., et al., “Expression of proteins in mammalian cells using vaccinia” In Current Protocols in Molecular Biology (F. M. Ausubel, et al. Eds.), Greene Publishing Associates & Wiley Interscience, New York (1991); Moss, B., et al., U.S. Pat. No. 5,135,855, issued Aug. 4, 1992), expression in bacteria (Ausubel, F. M., et al., CURRENT PROTOCOLS IN MOLECULAR BIOLOGY, John Wiley and Sons, Inc., Media Pa.; Clontech), expression in yeast (Rosenberg, S. and Tekamp-Olson, P., U.S. Pat. No. RE35,749, issued, Mar. 17, 1998, herein incorporated by reference; Shuster, J. R., U.S. Pat. No. 5,629,203, issued May 13, 1997, herein incorporated by reference; Gellissen, G., et al., Antonie Van Leeuwenhoek, 62(1-2):79-93 (1992); Romanos, M. A., et al., Yeast 8(6):423-488 (1992); Goeddel, D. V., Methods in Enzymology 185 (1990); Guthrie, C., and G. R. Fink, Methods in Enzymology 194 (1991)), expression in mammalian cells (Clontech; Gibco-BRL, Ground Island, N.Y.; e.g., Chinese hamster ovary (CHO) cell lines (Haynes, J., et al., Nuc. Acid. Res. 11:687-706 (1983); 1983, Lau, Y. F., et al., Mol. Cell. Biol. 4:1469-1475 (1984); Kaufman, R. J., “Selection and coamplification of heterologous genes in mammalian cells,” in Methods in Enzymology, vol. 185, pp 537-566. Academic Press, Inc., San Diego Calif (1991)), and expression in plant cells (plant cloning vectors, Clontech Laboratories, Inc., Palo-Alto, Calif, and Pharmacia LKB Biotechnology, Inc., Piscataway, N.J.; Hood, E., et al., J. Bacteriol. 168:1291-1301 (1986); Nagel, R., et al., FEMS Microbiol. Lett. 67:325 (1990); An, et al., “Binary Vectors”, and others in Plant Molecular Biology Manual A3:1-20 (1988); Miki, B. L. A., et al., pp. 249-265, and others in Plant DNA Infectious Agents (Hohn, T., et al., eds.) Springer-Verlag, Wien, Austria, (1987); Plant Molecular Biology: Essential Techniques, P. G. Jones and J. M. Sutton, New York, J. Wiley, 1997; Miglani, Gurbachan Dictionary of Plant Genetics and Molecular Biology, New York, Food Products Press, 1998; Henry, R. J., Practical Applications of Plant Molecular Biology, New York, Chapman & Hall, 1997), relevant portion incorporated herein by reference.

As used herein, the term “effective amount” or “effective dose” refers to that amount of the peptide or protein T cell epitopes of the invention sufficient to induce immunity, to prevent and/or ameliorate an infection or to reduce at least one symptom of an infection and/or to enhance the efficacy of another dose of peptide or protein T cell epitopes. An effective dose may refer to the amount of peptide or protein T cell epitopes sufficient to delay or minimize the onset of an infection. An effective dose may also refer to the amount of peptide or protein T cell epitopes that provides a therapeutic benefit in the treatment or management of an infection. Further, an effective dose is the amount with respect to peptide or protein T cell epitopes of the invention alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms, in this case, an infectious disease, and more particularly, an Alphavirus infection. The full prophylactic effect does not necessarily occur by administration of one dose, and may occur only after administration of a series of doses. Thus, a prophylactically effective amount may be administered in one or more administrations. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins), relevant portions incorporated herein by reference.

As used herein, the term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interferons, interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as peptide or protein T cell epitopes s of the invention, or can be administered separately. Either the protein or an expression vector encoding the protein can be administered to produce an immunostimulatory effect.

As used herein, in certain embodiments, the term “protective immune response” or “protective response” refers to an immune response mediated by antibodies against an infectious agent, which is exhibited by a vertebrate (e.g., a human), which prevents or ameliorates an infection or reduces at least one symptom thereof. Peptide and protein T cell epitopes of the invention can stimulate the production of antibodies that, for example, neutralize infectious agents, blocks infectious agents from entering cells, blocks replication of said infectious agents, and/or protect host cells from infection and destruction. In other embodiments, the term can also refer to an immune response that is mediated by T-lymphocytes and/or other white blood cells against an infectious agent, exhibited by a vertebrate (e.g., a human), that prevents or ameliorates Alphavirus infection or reduces at least one symptom thereof. Peptide and protein T cell epitopes of the invention can stimulate the T cell responses that, for example, neutralize infectious agents, kill virus infected cells, blocks infectious agents from entering cells, blocks replication of said infectious agents, and/or protect host cells from infection and destruction.

The terms “biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

As used herein, a “cell” refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

As used herein, the term “contacting” is used in accordance with its plain ordinary meaning and refers to the process of allowing at least two distinct species to become sufficiently proximal to react, interact or physically touch. It should be appreciated, however, the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be, for example, an amino acid sequence, protein, or peptide as provided herein and an immune cell, such as a T cell.

As used herein, a “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The terms “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The terms “subject” or “subject in need thereof” refers to a living organism who is at risk of or prone to having a disease or condition, or who is suffering from a disease or condition that can be treated by administration of a composition or pharmaceutical composition as provided herein. Non-limiting examples include humans and other primates, but also includes non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

The terms “disease” or “condition” refer to a state of being or health status of a patient or subject capable of being treated with a compound, pharmaceutical composition, or method provided herein. In embodiments, a patient or subject is human. In embodiments, the disease is Alphavirus infection. In certain alternative embodiments, the disease is Chikungunya virus infection. In still other embodiments, the disease is Chikungunya.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated or the disorder resulting from viral infection. Also, a therapeutic benefit is achieved with the eradication or amelioration of one or more of the physiological symptoms associated with viral infection or the underlying disorder such that an improvement is observed in the patient, notwithstanding that the patient may still be afflicted with the underlying disorder or may still be infected. For prophylactic benefit, the compositions may be administered to a patient at risk of viral infection, of developing a particular disease, or to a patient reporting one or more of the physiological symptoms of a disease, even though a diagnosis of this disease may not have been made. Treatment includes preventing the infection or disease, that is, causing the clinical symptoms of the disease not to develop by administration of a protective composition prior to infection or the induction of the disease; suppressing the disease, that is, causing the clinical symptoms of the disease or infection not to develop by administration of a protective composition after the inductive event or infection but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. “Treatment” can also refer to any of (i) the prevention of infection or reinfection, as in a traditional vaccine, (ii) the reduction or elimination of symptoms, and (iii) the substantial or complete elimination of the pathogen in question. Treatment may be affected prophylactically (prior to infection) or therapeutically (following infection).

In addition, in certain embodiments, “treatment,” “treat,” or “treating” refers to a method of reducing the effects of one or more symptoms of infection with an Alphavirus. Thus, in the disclosed method, treatment can refer to a 10%, 20%, 30%, 40%, 50%, 60%, 70%, 80%, 90%, or 100% reduction in the severity of an established infection, disease, condition, or symptom of the infection, disease or condition. For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition and/or complete prevention of infection. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

As used herein the terms “diagnose” or “diagnosing” refers to recognition of an infection, disease or condition by signs and symptoms. Diagnosing can refer to determination of whether a subject has an infection or disease. Diagnosis may refer to determination of the type of disease or condition a subject has or the type of virus the subject is infected with.

Diagnostic agents provided herein include any such agent, which are well-known in the relevant art. Among imaging agents are fluorescent and luminescent substances, including, but not limited to, a variety of organic or inorganic small molecules commonly referred to as “dyes,” “labels,” or “indicators.” Examples include fluorescein, rhodamine, acridine dyes, Alexa dyes, and cyanine dyes. Enzymes that may be used as imaging agents in accordance with the embodiments of the disclosure include, but are not limited to, horseradish peroxidase, alkaline phosphatase, acid phosphatase, glucose oxidase, β-galactosidase, β-glucoronidase or β-lactamase. Such enzymes may be used in combination with a chromogen, a fluorogenic compound or a luminogenic compound to generate a detectable signal.

The peptide(s) or protein(s) of the present invention can also be used in binding assays including, but are not limited to, immunoassays such as competitive and non-competitive assay systems using techniques such as western blots, radioimmunoassays, ELISA (enzyme linked immunosorbent assay), “sandwich” immunoassays, Meso Scale Discovery (MSD, Gaithersburg, Md.), immunoprecipitation assays, ELISPOT, precipitin reactions, gel diffusion precipitin reactions, immunodiffusion assays, agglutination assays, complement-fixation assays, immunoradiometric assays, fluorescent immunoassays, and protein A immunoassays. Such assays are routine and well known in the art (see, e.g., Ausubel et al., eds, 1994, Current Protocols in Molecular Biology, Vol. 1, John Wiley & Sons, Inc., New York, relevant portions incorporated herein by reference).

Radioactive substances that may be used as imaging agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

When the imaging agent is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

The term “adjuvant” refers to a compound that when administered in conjunction with the compositions provided herein including embodiments thereof, augments the composition's immune response. Generally, adjuvants are non-toxic, have high-purity, are degradable, and are stable.

Adjuvants can augment an immune response by several mechanisms including lymphocyte recruitment, stimulation of B and/or T cells, and stimulation of macrophages. The adjuvant increases the titer of induced antibodies and/or the binding affinity of induced antibodies relative to the situation if the immunogen were used alone. A variety of adjuvants can be used in combination with the agents provided herein including embodiments thereof, to elicit an immune response. Preferred adjuvants augment the intrinsic response to an immunogen without causing conformational changes in the immunogen that affect the qualitative form of the response. Preferred adjuvants include aluminum hydroxide and aluminum phosphate, 3 De-O-acylated monophosphoryl lipid A (MPL®) (see GB 2220211 (RIBI ImmunoChem Research Inc., Hamilton, Montana, now part of Corixa). STIMULON® QS-21 is a triterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, NY, 1995); U.S. Pat. No. 5,057,540), (Aquila BioPharmaceuticals, Framingham, MA). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent.

Other adjuvants contemplated for the invention are saponin adjuvants, such as STIMULON® (QS-21, Aquila, Framingham, MA) or particles generated therefrom such as ISCOMs (immunostimulating complexes) and ISCOMATRIX. Other adjuvants include RC-529, GM-CSF and Complete Freund's Adjuvant (CFA) and Incomplete Freund's Adjuvant (IFA). Other adjuvants include cytokines, such as interleukins (e.g., IL-1 α and β peptides, IL-2, IL-4, IL-6, IL-12, IL-13, and IL-15), macrophage colony stimulating factor (M-CSF), granulocyte-macrophage colony stimulating factor (GM-CSF), tumor necrosis factor (TNF), chemokines, such as MIP1α and β and RANTES. Another class of adjuvants is glycolipid analogues including N-glycosylamides, N-glycosylureas and N-glycosylcarbamates, each of which is substituted in the sugar residue by an amino acid, as immuno-modulators or adjuvants (see U.S. Pat. No. 4,855,283). Heat shock proteins, e.g., HSP70 and HSP90, may also be used as adjuvants.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration. As used herein, the terms “pharmaceutically acceptable” or “pharmacologically acceptable” refer to a material which is not biologically or otherwise undesirable, i.e., the material may be administered to an individual in a formulation or composition without causing any unacceptable biological effects or interacting in a deleterious manner with any of the components of the composition in which it is contained.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like., that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

The present invention describes methods utilizing and compositions comprising or expressing T cell epitopes, T cell epitope-containing peptides, and T cell epitope-containing proteins associated with binding to a subset of the naturally occurring MHC Class II and/or MHC Class I molecules within the human population. Compositions comprising or expressing one or more of the disclosed peptides (e.g., the amino acid sequences set forth in Table 1 (SEQ ID NOS: 1 to 150)) or polynucleotides encoding the same, covering different HLA Class II and/or MHC Class I alleles, capable of generating a treatment acting broadly on a population level are disclosed herein. As the antigen repertoire of MHC Class I and MHC Class II alleles varies from one individual to another and from one ethnic population to another, it is challenging to provide vaccines or peptide or epitopes-based immunotherapies that can be offered to subjects of any geographic region in the world or provide sufficient protection against infection across a wide segment of the populations unless numerous epitopes or peptides are included (e.g., in a vaccine). Taking into consideration the need for a single vaccine formulation that can provide protection across populations, if it desirable to provide a treatment containing or expressing proteins, peptides or epitopes that will provide protection against infection amongst the majority of the worldwide population. Also, taking into consideration the enormous costs and risks in the clinical development of new treatments and the increasing demands from regulatory bodies to meet high standards for toxicity testing, dose justification, safety and efficacy trials, it is desirable to provide treatments containing or expressing as few peptides as possible, but at the same time to be able to treat the majority of subjects in a worldwide population with a single immunotherapy. Such a product should comprise as a first requirement an expression or inclusion of combination of epitopes or peptides that are able to bind the worldwide MHC Class I and/or MHC Class II allele repertoire, and the resulting peptide-MHC complexes should as a second requirement be recognized by the T cells of the subject so as to induce the desired immunological reactions.

It is an object of claims of the present invention to provide improved epitope or peptide combinations for modulating an immune response, for treating a subject for an infection or aberrant immune response, and for use in diagnostic methods and kits comprising such peptide combinations. It is another object of the invention to provide epitope or peptide combinations exhibiting very good HLA Class I and Class II coverage in a worldwide population and being immunologically potent in a worldwide population. It is another object of the invention to provide epitope or peptide combinations having good cross reactivity to other strains, including co-circulating strains (for example, mutants) of Alphavirus, including Chikungunya virus, etc. It is another object of the invention to provide epitope or peptide combinations of a relatively small number of epitopes or peptides yet obtaining at least 70%, and more preferably around 90-100% donor coverage in a donor cohort representative of a worldwide population. In certain embodiments, this is achieved by selecting one or more immunodominant and/or immunoprevalent proteins (e.g., a Chikungunya viral protein) or subsequences, portions, homologues, variants or derivatives thereof for use in the methods and compositions of the present disclosure, wherein said immunodominant and/or immunoprevalent proteins or subsequences, portions, homologues, variants or derivatives thereof comprise two or more epitopes that are immunodominant and/or immunoprevalant. In some embodiments, the two or more epitopes comprise two to ten epitopes and/or polynucleotides encoding the same. Another object of the invention is to provide epitope combinations which are so immunologically potent that even at very low doses of epitopes, the percentage of responding donors can be retained at a very high level in a donor cohort representative of a worldwide population. Another object of the invention is to provide epitope combinations which have minor risk of inducing IgE-mediated adverse events. An additional object of the invention is to provide proteins, peptides, or nucleic acids containing or expressing epitopes or combinations of such proteins, peptides or nucleic acids which have a sufficient solubility profile for being formulated in a pharmaceutical product, preferably which have acceptable estimated in vivo stability. One further objective of the invention is to select epitopes for use in the compositions and methods described herein, based on one or both of their immunodominance or immunoprevalence. A still further object of the invention is to select such epitopes and epitopes combinations not only in accordance with those embodiments previously described, but also those epitopes and epitope combinations capable of eliciting a B cell response and T cell response (e.g., selecting one or more peptides for use in the methods and compositions described herein capable of generating a T cell and antibody response in a subject).

Provided herein are methods and compositions for diagnosing, treating, and immunizing against an Alphavirus, including methods and compositions of detecting an immune response or immune cells relevant to an Alphavirus infection. These methods and compositions include vaccines, diagnostics, therapies, reagents and kits, for modulating, eliciting, or detecting T cells responsive to one or more Alphavirus peptides or proteins. The proteins and peptides described herein comprise, consist of, or consist essentially of: one or more amino acid sequences selected from any sequence set forth in Table 1 (SEQ ID NOS:1 to 150), or a subsequence, portion, homologue, variant or derivative thereof, a fusion protein comprising one or more amino acid sequences selected from any sequence set forth in Table 1 (SEQ ID NOS:1 to 150); a pool of 2 or more peptides selected from the amino acid sequences set forth in Table 1 (SEQ ID NOS:1 to 150), or a polynucleotide that encodes one or more peptides or proteins, comprising, consisting of, or consisting essentially of an amino acid sequence selected from any sequence set forth in Table 1 (SEQ ID NOS:1 to 150), or a subsequence, portion, homologue, variant or derivative thereof. In certain preferred embodiments, the Alphavirus is one or more of Chikungunya virus or a variant thereof. Further description and embodiments of such methods and compositions are provided in the definitions provided herein, and a person skilled in the art will recognize that the methods and compositions can be embodied in numerous variations, changes, and substitutions or as may occur to or be understood by one skilled in the art without departing from the invention.

Study cohort. To identify T cell epitopes in CHIKV infection, the inventors collected PBMCs from 17 patients in Colombia who had been clinically diagnosed with CHIKV using an epidemiological diagnosis criterion. A detailed description of the diagnostic criteria has been added to the methods. Infections were later confirmed by the presence of CHIKV-IgG antibodies. Both sexes were represented [M:F, 6:11] and the mean age at the time of sample collection was 46.8 years. Samples were collected on an average 6.3 years post-infection and all individuals displayed symptoms of arthralgia at the time of sample collection.

36 donors were enrolled in Colombia who had been diagnosed with CHIKV during the 2014-2015 epidemic and five uninfected CHIKV-seronegative donors. The criteria of a positive CHIKV diagnosis included: 1) whether the symptoms matched with CHIKV, 2) whether the patient lived in or visited a region where CHIKV had been detected using a RT-PCR assay, 3) whether other of the patient's household or other residents in the neighborhood had been diagnosed with CHIKV, 4) whether the infections with dengue virus, which often co-circulates with CHIKV and has similar clinical presentation, has been ruled out through laboratory tests such as ELISA and rapid tests. Not all patient samples were subjected to laboratory tests to confirm CHIKV infection. Local medical professionals determined if the epidemiological link based on the criteria mentioned above was considered sufficient to diagnose a case as CHIKV positive. For the purpose of these studies, the inventors confirmed infection by measuring the levels of CHIKV-IgG antibodies using the serum of all individuals.

At the time of enrollment in the study, all individual donors provided informed consent that their samples could be used for any future studies, including this study. All blood and serum samples were anonymized and given a code number. In addition to collecting blood and serum samples, regarding the date of their CHIKV diagnosis, the date of symptom onset, the date of sample collection, their age, their gender, and the specific symptoms at the time of CHIKV diagnosis and sample collection was also collected for each participant. The donors were of both sexes were represented. All 36 donors, at the time of collection, still exhibited chronic symptoms resulting from previous CHIKV infection.

PBMC isolation. Whole blood samples were collected from CHIKV donors at a site in Colombia. Large volumes of the donors' blood were collected in sterile bags which contained 3.27 g citric acid, 26.3 g NaH₂-citrate and 2.22 g NaHCO3 per liter (Fresenius Kabi, Fresenius HemoCare, Brasil Ltd, Brasil) and then diluted by 50% v/v with RPMI-1640 media (31800-105, Gibco, USA) containing 0.2% w/v NaHCO3 and 34 ml volumes were gentle layered into multiple sterile 50 ml tubes (430290, Corning, USA) containing 12 ml of sterile endotoxin-tested Ficoll-Paque PLUS (1.077 g/ml density) (GE Healthcare Biosciences, Sweden). After centrifugation at 500×g, the buffy-coat peripheral blood mononuclear cells (PBMCs) were collected from immediately above the Ficoll-Paque-plasma interface, diluted with 50% v/v RPMI 1640 medium and again centrifuged at 500×g to pellet the PBMCs. These PBMCs were then re-suspended in large volumes of RPMI 1640 medium containing 5% v/v fetal calf serum (FCS) (F-0926, Sigma, USA) and centrifuged against at 500×g and after a further wash their PMBCs were re-suspended in ice-cold 10% dimethyl sulfoxide (D-2650, Sigma, USA) in FCS at approximately 20×10⁶ PBMCs/ml and 1 ml aliquots contained in 1.5 ml polypropylene cryo-vials (5000-1020: Nalgene System 100, Thermo Scientific, USA) were slowly (−1° C./min) frozen down to −80° C. in a isopropanol alcohol-walled freezing unit (Nalgene Mr. Frosty: C-1562 Sigma-Aldrich, USA) before being transferred to 25-vial boxes in liquid nitrogen.

Peptide pools. Since only a limited number of full-length sequences of CHIKV polyprotein were available from NIAID Virus Pathogen Database and Analysis Resources (ViPR) database, the inventors made two separate consensus alignments for structural and non-structural polyprotein sequences and retrieved 257 and 350 full-length CHIKV structural and non-structural polyprotein sequences from ViPR using the following query: Chikungunya virus, Gene product name: structural OR non-structural polyprotein, Remove duplicate sequences. Unresolved sequences were removed.

The number of sequences available varied as a function of geographic locations. To ensure balanced representation, the number of isolates by geographical region was limited to a maximum of 10. In total, 158 structural and 61 non-structural sequences were selected. For each polyprotein, sequences were aligned using MUSCLE, and consensus sequences were BLASTed to identify a representative isolate (GenBank ID: AQX78118.1 and AQX78116.1), using tools in ViPR. To account for variants to the consensus sequences, the inventors additionally synthesized 105 and 146 amino acid variants with a frequency above 10% from structural and non-structural consensus sequences, respectively.

Based on the consensus sequence and the variants, the inventors synthesized 15-mer peptides overlapping by 10 residues. Together, peptides that spanned the length of the CHIKV proteome were synthesized, resuspended in dimethyl sulfoxide (DMSO), and divided into 11 pools. Since the nsP2 megapool was too large, it was split into two pools (nsP2_1 and nsP2_2) of 117 and 81 peptides, respectively.

This megapool approach has been used previously to simultaneously screen a large number of epitopes. In this approach, large numbers of different epitopes are solubilized, pooled, and re-lyophilized to avoid cell toxicity problems associated with high concentrations of DMSO typically encountered when pooling after a single solubilization step. These MPs have been used in several indications, including SARS-CoV-2 [28], allergies [30], tuberculosis [31], pertussis[32], tetanus [33] and DENV for both CD4⁺ and CD8⁺ T cell epitopes [34,35].

Mesopools were made from individual megapools and consisted of 9-11 peptides. These number of mesopools per megapool ranges from 7 to 16, depending on the size of the megapools. The mesopool screening approach decreases the number of individual peptides needed to be tested when the pool is positive.

For epitope megapools, reference and variant epitopes were synthesized, resuspended in DMSO post lyophilization and divided into structural and non-structural megapools, which were used to validate epitope megapools in an independent cohort. Each pool was tested 6-19 times.

Activation induced Marker assay and Intracellular staining assay. PBMCs were thawed in 10 mL of RPMI 1640 (Corning) supplemented with 5% human AB serum (GeminiBio), penicillin [100 IU/mL], streptomycin [100 μg/mL](GeminiBio), and 2 mM L-glutamine (Gibco), and in the presence of benzonase (20 μL/10 mL). PBMCs were then plated at 1×10 cells per well in 96-well U bottom plates and stimulated separately with each CHIKV-specific megapool (MP) [1 μg/mL] or CHIKV structural and non-structural epitope megapool. An equimolar amount of DMSO was used for negative control. Stimulation with phytohemagglutinin (PHA, Roche) [1 μg/mL] and a combined CD4 and CD8 CMV MP were used for positive controls [1 μg/mL]. For samples that were tested for epitope identification, cells were stained for detection of activation-induced markers after 24 hours of stimulation.

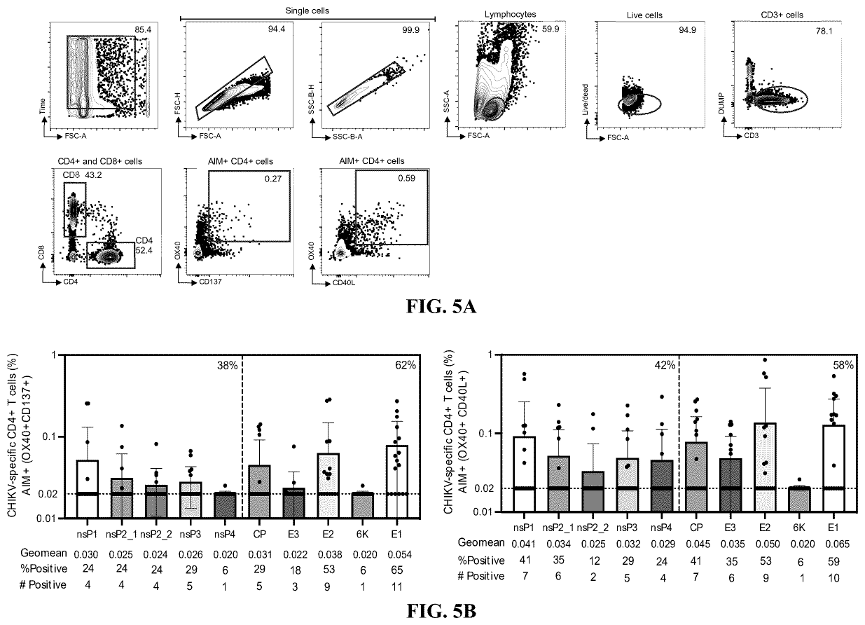

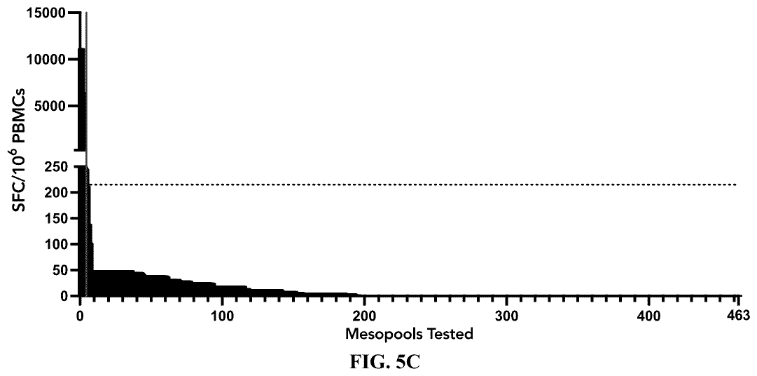

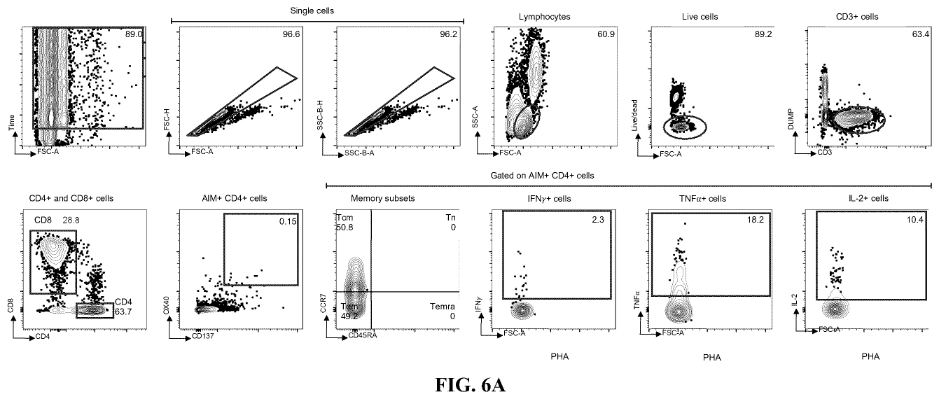

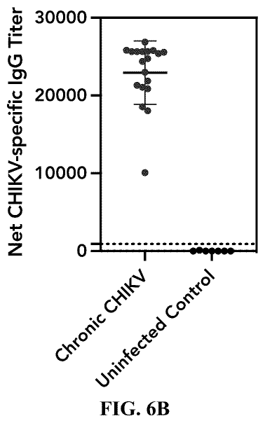

For samples that were tested for epitope validation, the inventors proceeded with intracellular staining assay post-stimulation. After 20 h post-stimulation, Golgi-Plug and Golgi-Stop were added to the culture, in addition to anti-CD69 and anti-CD137 Abs. Cells were then washed, incubated with BD human FC block, and stained with LIVE/DEAD marker in the dark for 15 minutes. After incubation, cells were washed, surface stained in the dark for 30 minutes at 4° C., and then fixed with 1% of paraformaldehyde (Sigma-Aldrich, St. Louis, MO). Subsequently, cells were permeabilized and stained with intracellular antibodies in the dark for 30 minutes at RT. All samples were acquired on Cytek Aurora. Antibodies used in the initial AIM screening assay are listed in Table 4 and the antibodies used in the AIM/ICS validation assay are listed in Table 5. A representative gating strategy for the AIM assay is shown in supplemental FIG. 5A and for the combined AIM/ICS assays in FIG. 6A.

Antigen-specific CD4⁺ T cells were measured as a percentage of AIM+ (OX40⁺CD137⁺ and OX40+CD40L+) CD4⁺ T cells. The stimulation index (SI) was calculated by dividing the percentage of stimulated samples by those of the DMSO control. Limit of detection (LOD) was calculated based on two-fold geometric 95% confidence interval of all negative values. Limit of sensitivity (LOS) was calculated using the median 2-fold standard deviation of all DMSO values. Response <LOD (0.02%) and SI<2, after background subtraction, was normalized to the LOD. SI>2 and response >LOS (0.02%) was considered a positive response for antigen-specific CD4⁺ T cells. SI>2 and response >LOS (0.04% for antigen-specific CD4⁺ T cells) and SI>2 was considered a positive response for combined AIM/ICS assay.

T cell lines. CD4⁺ T cells were cultured at 37° C. and 5% CO2 in RPMI 1640 supplemented with 5% human serum at a density of 2×10⁶ cells per well in 6-well plates. The cells were stimulated individually with each megapool that the donor was AIM positive for. Interleukin 2 (IL-2; 10 U/ml) was added every 3 to 4 days until cell harvest at day 14 (for mesopool evaluation) or day 17 (for individual peptide evaluation).

FluoroSpot assay. Following in vitro expansion at day 14, CHIKV-specific responses were assessed by gamma interferon (IFNγ) Fluorospot to detect responses to mesopools. At day 17, positive pools were deconvoluted by Fluorospot using individual CHIKV peptides (10 μg/ml) contained in the positive pool. Briefly, polyvinylidene fluoride (PVDF) plates (Millipore) were coated with anti-human IFNγ (1-D1K; Mabtech), and cells were plated in triplicate at 5×10⁴ cells per well. Cells were stimulated with either peptide pools (1 μg/ml) or individual peptides (10 μg/ml) in 0.1 ml complete RPMI and incubated for 24 hours at 37° C. and 5% CO2. Following stimulation, cells were discarded, and plates were incubated with biotinylated IFNγ monoclonal antibodies (MAb) (7-B6-1; Mabtech) for 2 hours at 37° C. and developed as described previously [36]. Positive responses were identified as those having >215 spot-forming cells (SFCs) per million cells, a stimulation index of >2, and a p-value of <0.05 when compared to unstimulated cells using a t-test as previously described [37].

Serologic assays. Site-specifically biotinylated CHIKV E1 DIII and Halo-tag control antigens were coupled to unique MagPlex®-Avidin microspheres at a concentration of 5 μg of antigen per 10⁶ beads in assay buffer (1% BSA+phosphate-buffered saline (PBS), pH 7.4) for 1 h at 37° C. with shaking at 700 rpm as described before[38]. Antigen coupled beads were washed and aliquoted 2,500 beads per antigen per well into a 96-well assay plate. Heat-inactivated (56° C. for 30 minutes) human serum samples diluted at 1:500 in assay buffer were incubated with beads for 1 hour at 37° C. with shaking at 700 rpm. After washing the beads with assay buffer, PE conjugated goat anti-human IgG Fc secondary Ab was added at 6 μg/ml (Southern Biotech, catalog: 2014-09) and incubated for 1 hour at 37° C. with shaking at 700 rpm. Beads were washed and resuspended in 100 μL assay buffer for fluorescence analysis using the Luminex 200 system.

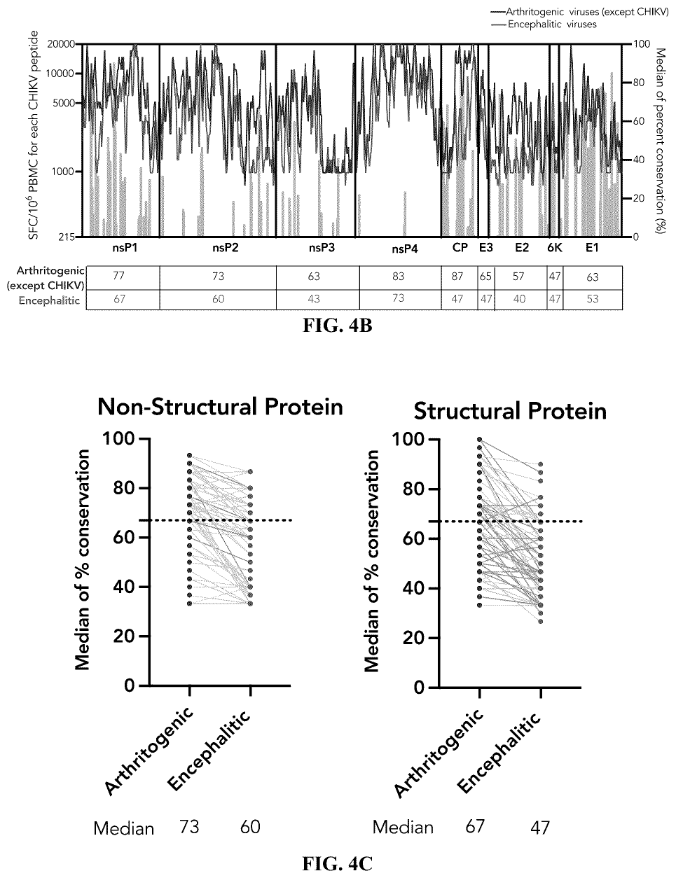

Analysis of conservation of epitopes and non-epitopes in alphaviruses. Sequences of all alphaviruses were extracted from Bacterial and Viral Bioinformatics Center (BV-BRC) based on sequence names provided in the phylogenetic tree in FIG. 4A. Sequences were aligned using the online MAFFT version 7 alignment tool (mafft.cbrc.jp/alignment/server/index.html) and Neighbor-joining method (with default settings) was used to create a tree. The tree was visualized using Interactive Tool of Life (ITOL) web server. To calculate conservation, viruses were divided into arthritogenic, and encephalitic subgroups based on their main pathogenic characteristics. IEDB epitope conservancy tool, with the threshold of 10%, was used to determine percent identity of each CHIKV peptide in the extracted alphaviral sequences. Median of percent conservation was calculated separately for encephalitic and arthritogenic alphaviruses. The analysis was performed separately for structural and non-structural polyproteins for each alphavirus.

Quantification and statistical analysis. Data and statistical analyses were done in FlowJo 10.8.1 and GraphPad Prism 9 (La Jolla, CA) unless otherwise stated. The statistical details of the experiments are provided in the respective figure legends and in method details. Data plotted in linear scale were expressed as Mean±Standard Deviation (SD). Data plotted in logarithmic scales were expressed as Geometric Mean±Geometric Standard Deviation (SD). Mann-Whitney tests was applied for unpaired non-parametric comparisons. Kruskal-Wallis test adjusted with Dunn's test for multiple comparisons was used to compare multiple groups.

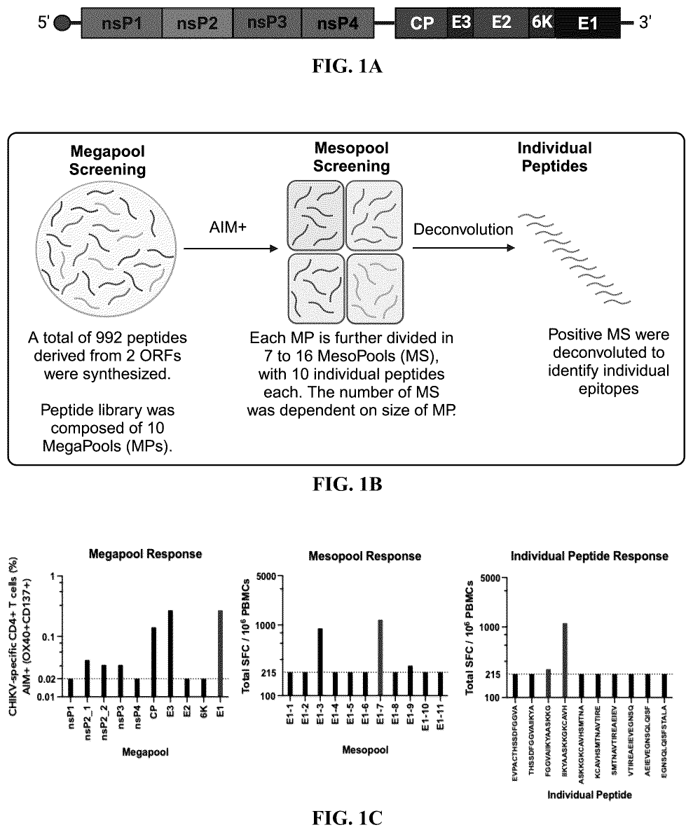

Experimental design for proteome-wide screen of CHIKV human T cell epitope. Currently, T cell epitopes recognized during acute CHIKV infection and chronic CHIKVD in humans are unknown. Here, the inventors used a previously described megapool screening approach where the inventors synthesized 15-mer overlapping peptides for four non-structural CHIKV proteins (nsP1, nsP2, nsP3, nsP4) and five structural CHIKV proteins (CP, E3, E2, 6K, E1), which the inventors refer to as megapools (MPs) (FIG. 1A) [20,25]. Overall, MPs consisted of total 992 peptides, which comprised of 741 reference peptides and 251 variant peptides. The criteria to determine variant peptides is detailed in the methods. Each of the nine MPs were further divided into smaller pools of 9-10 individual peptides, which are refer to as mesopools.

To identify the global pattern of recognition, the inventors first performed 24-hour ex vivo restimulation of PBMCs with each CHIKV protein MP (nsP1, nsP2_1, nsP2_2, nsP3, nsP4, CP, E3, E2, 6K, E1) for the entire study cohort. As described before, negligible CD8+ T cell responses were detected, hence, the inventors focused epitope identification efforts on CD4⁺ T cells. Chikungunya-specific CD4⁺ T cells were identified in an activation induced marker assay (AIM assay), using the upregulation of OX40+CD137+ or OX40+CD40L+ markers, both of which are commonly used to measure antigen-specific CD4⁺ T cell responses [26-28]. As previously described, responses greater than the limit of sensitivity (LOS; calculated based on median two-fold standard deviation of T cell reactivity in negative controls) and stimulation index (S.I.) greater than two were considered positive. CHIKV-specific CD4⁺ T cell responses were detected in 88% of (15/17) donors with responses directed against an average of three proteins (FIG. 5B). Out of 170 unique donor-protein combinations tested, total 47 positive responses were detected marked by upregulation of OX40+CD137+ and 57 positive responses marked by upregulation of OX40+CD40L+. 33 responses were positive in both. CD4⁺ T cell responses to structural proteins and non-structural proteins accounted for 62% (29/47) and 38% (18/47) of the total CHIKV-specific CD4⁺ T cell responses per protein, respectively, based on OX40+CD137+ upregulation, and 58% (33/57) and 42% (24/57) of total CHIKV-specific CD4⁺ T cell responses, respectively based on OX40+CD40L+ upregulation (FIG. 5B).

All positive responses for each donor in the AIM assay were restimulated in an in vitro T cell assay pending cell availability. If the number of more donor-protein combinations succeeded the number of available cells, the strongest AIM+ responses were selected for restimulation. After 14 days of restimulation, each T cell culture was tested against mesopools from the corresponding MP in a FluoroSpot assay. Overall, a total of 795 mesopools were tested and 293 (37%) mesopools were positively identified. Finally, positive mesopool responses were deconvoluted for each donor to identify the individual epitope that is recognized. A total of 1662 unique peptide conditions were deconvoluted, and 123 reference epitopes and 24 variant epitopes were identified (Table 3). The amino acid change is in bold.

TABLE 3
Variant Epitopes.

| Ref Sequence | SEQ ID NO: | Variant Epitope | SEQ ID NO: | Megapool | Screened in |
|---|---|---|---|---|---|
| PRGAIKVTAQPTDHV | 122 | PRGAIKVTAQLTDHV | 116 | nsP2_2 | 1/2 |
| KVTAQPTDHVVGEYL | 123 | KVTAQLTDHVVGEYL | 138 | nsP2_2 | 1/2 |
| PTDHVVGEYLVLSPQ | 92 | LTDHVVGEYLVLSPQ | 118 | nsP2_2 | 1/2 |
| FSNYSESEGDRELAA | 124 | FSNYTESEGDRELAA | 139 | nsP3 | 1/1 |
| HPPISFGASSETFPI | 125 | HPPISFGAPSETFPI | 120 | nsP3 | 1/1 |
| NEGEIESLSSELLTF | 126 | NDGEIESLSSELLTF | 121 | nsP3 | 1/1 |
| NRRYQPRPWTPRPTI | 34 | NRRYQPRPWTPRSTI | 57 | CP | 1/4 |
| RPRPQRQAGQLAQLI | 127 | RPRPQRKAGQLAQLI | 58 | CP | 1/4 |
| LAQLISAVNKLTMRA | 16 | LAQLISAVNKLTMRV | 59 | CP | 4/4 |
| SAVNKLTMRAVPQQK | 7 | SAVNKLTMRVVPQQK | 60 | CP | 2/4 |
| KPAQKKKKPGRRERM | 128 | KPVQKKKKPGRRERM | 61 | CP | 1/1 |
| DNVMRPGYYQLLQAS | 129 | DNVMSPGYYQLLQAS | 62 | JE3 | 1/1 |
| MSQQSGNVKITVNGQ | 130 | MSQQSGNVKITVNSQ | 63 | E2 | 1/3 |
| GNVKITVNGQTVRYK | 27 | GNVKITVNSQTVRYK | 64 | E2 | 3/3 |
| TTTDKVINNCKVDQC | 28 | ITTDKVINNCKIDQC | 65 | E2 | 1/1 |
| KVDQCHAAVTNHKKW | 29 | KIDQCHAAVTNHKKW | 66 | E2 | 1/1 |
| VMHKKEVVLTVPTEG | 131 | VTHKKEIRLTVPTEG | 67 | E2 | 1/1 |
| EVVLTVPTEGLEVTW | 132 | EIRLTVPTEGLEVTW | 67 | E2 | 2/2 |
| LLSLICCIRTAKAAT | 133 | LLSLLCCIRTAKAAT | 69 | E2 | 1/2 |
| EQQPLFWLQALIPLA | 134 | EQQPLFWMQALIPLA | 140 | 6K | 2/3 |
| TQLVLQRPAVGTVHV | 39 | TQLVLQRPSAGTVHV | 70 | E1 | 1/1 |
| ATNPVRAVNCAVGNM | 135 | ATNPVRAMNCAVGNM | 71 | E1 | 3/5 |
| AVGNMPISIDIPEAA | 136 | AVGNMPISIDIPDAA | 72 | E1 | 2/4 |
| PISIDIPEAAFTRVV | 137 | PISIDIPDAAFTRVV | 73 | E1 | 2/4 |

A summary of the screening strategy is shown in FIG. 1B and representative data from one donor is shown in FIG. 1C. The representative donor recognized six MPs (nsP2_1, nsP2_2, nsP3, CP, E3 and E1) in the AIM assay, which were then screened with mesopools. From the E1 mesopools, the donor recognized three mesopools (E1-3, E1-7 and E1-9). The E1-7 mesopool, among other pools, was then deconvoluted where two epitopes from the pool were recognized by the donor. The threshold of positivity of 215 SFC/10⁶ PBMCs for the FluoroSpot assay was determined by measuring responses to all CHIKV mesopools in six CHIKV-seronegative donors (FIG. 5C). As the variant peptide overlap with the reference peptides, the inventors chose to focus on only reference epitopes for further analysis.

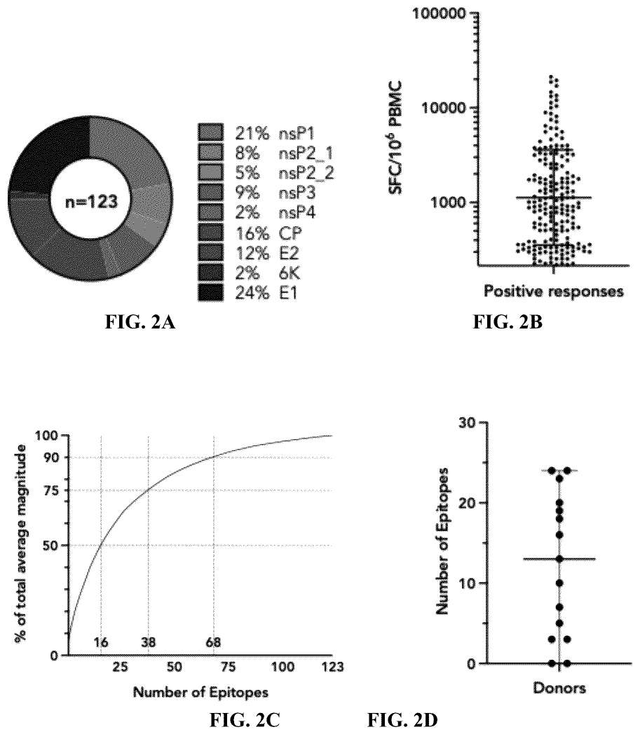

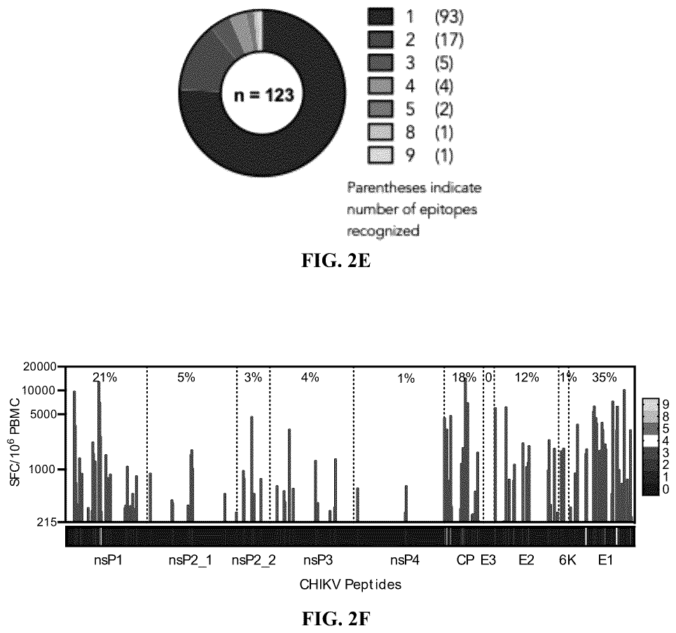

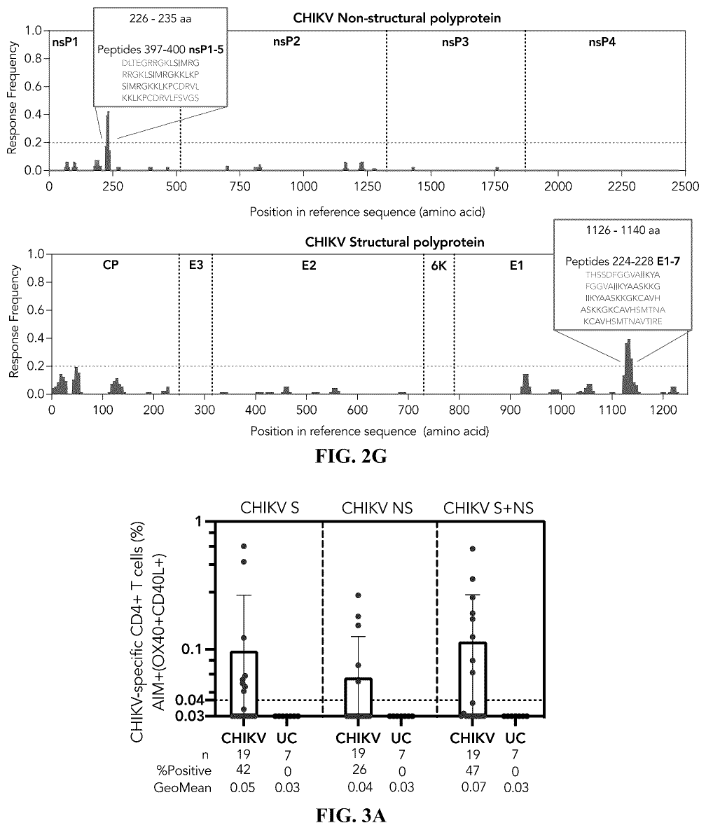

Immunodominant proteins in CHIKV proteome recognized by CD4 T cells. Post-deconvolution, the inventors identified a total of 123 epitopes from the reference genome (FIG. 2A). Majority of reference epitopes were from E1 (240%), nsP1 (210%) and CP (16%) proteins with few epitopes in nsP4 (2%) and 6K (2%). No epitopes were identified in the E3 protein. In addition, the magnitude of response from epitopes identified from the reference sequence varied widely with average response at 1128 SFC/10⁶ PBMCs (FIG. 2B). Interestingly, only 38 epitopes accounted for 75% of the total magnitude of IFNγ response from all donors tested (FIG. 2C and Table 2).

TABLE 2
38 epitopes accounted for 75% of the total magnitude of IFNy response from
all donors tested.

| | | | | | Average |
|---|---|---|---|---|---|
| | | | | Screened In | IFNY |
| | | | | (Positive | Response |
| | | | | donors/Total | (SFC per 106 |
| Ranking | Sequence | SEQ ID NO: | CHIKV MP | donors tested) | PBMCs) |
| 1 | KPAHVKGTIDNADLA | 14 | CP | 1/5 | 14653 |
| 2 | DLTEGRRGKLSIMRG | 84 | nsP1 | 1/6 | 13120 |
| 3 | SAEFRVQVCSTQVHC | 52 | E1 | 1/2 | 10413 |
| 4 | DPDSTILDIGSAPAR | 74 | nsP1 | 1/3 | 9973 |
| 5 | FTRVVDAPSLTDMSC | 45 | E1 | 1/2 | 7460 |
| 6 | RRGKLSIMRGKKLKP | 85 | nsP1 | 5/5 | 7248 |
| 7 | KLAFKRSSKYDLECA | 15 | CP | 1/2 | 7113 |
| 8 | SSAWTPFDNKIVVYK | 34 | E1 | 1/1 | 6447 |
| 9 | IIKYAASKKGKCAVH | 47 | E1 | 9/10 | 6393 |
| 10 | ADAERAGLFVRTSAP | 23 | E2 | 2/3 | 6307 |
| 11 | FNVYKATRPYLAHCP | 141 | E2 | 2/6 | 6137 |
| 12 | IVGPMSSAWTPFDNK | 33 | E1 | 1/1 | 5573 |
| 13 | LAQLISAVNKLTMRA | 6 | CP | 5/5 | 4889 |
| 14 | LGRYDLVVINIHTPF | 98 | nsP2_2 | 1/1 | 4760 |
| 15 | IVVYKGDVYNMDYPP | 36 | E1 | 1/1 | 4633 |
| 16 | MEFIPTQTFYNRRYQ | 1 | CP | 2/6 | 4577 |
| 17 | TQLVLQRPAVGTVHV | 39 | E1 | 1/4 | 4013 |
| 18 | GDVYNMDYPPFGAGR | 37 | E1 | 1/1 | 3940 |
| 19 | SPYVKCCGTAECKDK | 142 | E1 | 1/1 | 3807 |
| 20 | ILDIGSAPARRMMSD | 75 | nsP1 | 1/3 | 3693 |
| 21 | PRPWTPRPTIQVIRP | 4 | CP | 4/6 | 3297 |
| 22 | QSLNHLFTAMDSTDA | 143 | nsP3 | 1/2 | 3293 |
| 23 | QRPAVGTVHVPYSQA | 40 | E1 | 1/4 | 3273 |
| 24 | FGGVAIIKYAASKKG | 46 | E1 | 8/10 | 3250 |
| 25 | ISATAMSWVQKITGG | 55 | E1 | 2/2 | 3230 |
| 26 | PFDNKIVVYKGDVYN | 35 | E1 | 1/1 | 3060 |
| 27 | SIMRGKKLKPCDRVL | 86 | nsP1 | 4/5 | 2670 |
| 28 | ILYYYELYPTMTVVV | 144 | E2 | 1/2 | 2420 |
| 29 | VGFDTTPFMYNAMAG | 81 | nsP1 | 1/3 | 2267 |
| 30 | GNVKITVNGQTVRYK | 27 | E2 | 2/3 | 2205 |
| 31 | PSGFKYWLKERGASL | 42 | E1 | 3/4 | 2131 |
| 32 | HAAVTNHKKWQYNSP | 30 | E2 | 3/3 | 2036 |
| 33 | GKVTGYACLVGDKVM | 11 | CP | 2/5 | 1917 |
| 34 | ALIVLCNCLRLLPCC | 145 | 6K | 1/3 | 1893 |
| 35 | CARRRCITPYELTPG | 146 | E2 | 1/2 | 1887 |
| 36 | YWLKERGASLQHTAP | 43 | E1 | 3/4 | 1856 |
| 37 | EFASAYRAHTASASA | 32 | E1 | 4/4 | 1853 |
| 38 | FFNMMQMKVNYNHNI | 147 | nsP2_1 | 1/4 | 1800 |

On an average, the inventors detected epitopes in 13/15 donors, whereby each donor identified on an average 12 epitopes with responses ranging from no epitopes identified to 25 epitopes identified (FIG. 2D). As expected in analyzing a heterogenous MHC donor cohort, 75% of epitopes were only identified in one donor (FIG. 2E). Interestingly, four epitopes were more frequently recognized, with two epitopes—E1₁₁₂₁₋₁₁₃₅ (FGGVAIIKYAASKKG)(SEQ ID NO:46) and E11₁₂₆₋₁₁₄₀ (IIKYAASKKGKCAVH) (SEQ ID NO:47)—being recognized by the eight and nine donors, respectively (FIG. 2E). Both E1₁₁₂₁₋₁₁₃₅ and E11₁₂₆₋₁₁₄₀ epitopes shared the same eight donors. The other two commonly recognized epitopes—CP₄₁₋₅₅ (LAQLISAVNKLTMRA)(SEQ ID NO:6) and nsP1₂₂₁₋₂₃₅ (RRGKLSIMRGKKLKP)(SEQ ID NO:85)—were recognized by five donors each. Additionally, all four of the commonly recognized epitopes also elicited a high magnitude of response (FIG. 2F). Overall, non-structural and structural proteins accounted for 34% and 66% of total magnitude of the response, respectively, with the highest magnitude of responses against epitopes in E1 (35%), nsP1 (21%) and CP (18%). Interestingly, in addition to the having the highest number of epitopes and the highest frequency of response, E1 protein accounted for over one-third of the overall magnitude of the response.

Next, to identify the immunodominant regions in CHIKV proteome, the inventors mapped the epitopes to the CHIKV proteome using ImmunomeBrowser from Immune Epitope Database (IEDB) and calculated the lower 95% confidence interval of the response frequency of each peptide in structural and non-structural polyproteins separately (number of donors with positive responses divided by total number of donors based on mesopool response; if a donor was tested negative to a mesopool it was counted as part of the total response). Two regions, nsP1(226-235aa, which includes nsP1₂₂₁₋₂₃₅ peptide) and E1(1126-1140aa, which includes E1₁₁₂₁₋₁₁₃₅ and E11₁₂₆₋₁₁₄₀ peptides), reached above the threshold of positivity (FIG. 2G).

Overall, the inventors identified 123 CHIKV epitopes of CD4⁺ T cells, 38 of which elicited high levels of IFNγ response. In addition, the inventors identified two immunodominant regions in nsP1 and E1 proteins that were recognized by individuals with chronic CHIKVD.

Development and validation of a new CHIKV CD4⁺ T cell epitope megapool.

Next, the inventors wanted to create a CHIKV-specific epitope MP that can be used to study CD4⁺ T cell responses if only small amounts of blood are available, as typical in pediatric or clinical studies. The inventors created separate structural and non-structural MPs that consisted of 123 reference epitopes and 24 variant epitopes that the inventors had previously identified. The structural epitope MP, referred to as CHIKV S, and the non-structural epitope MP, referred to as CHIKV NS, consisted of 84 and 63 epitopes each, respectively.

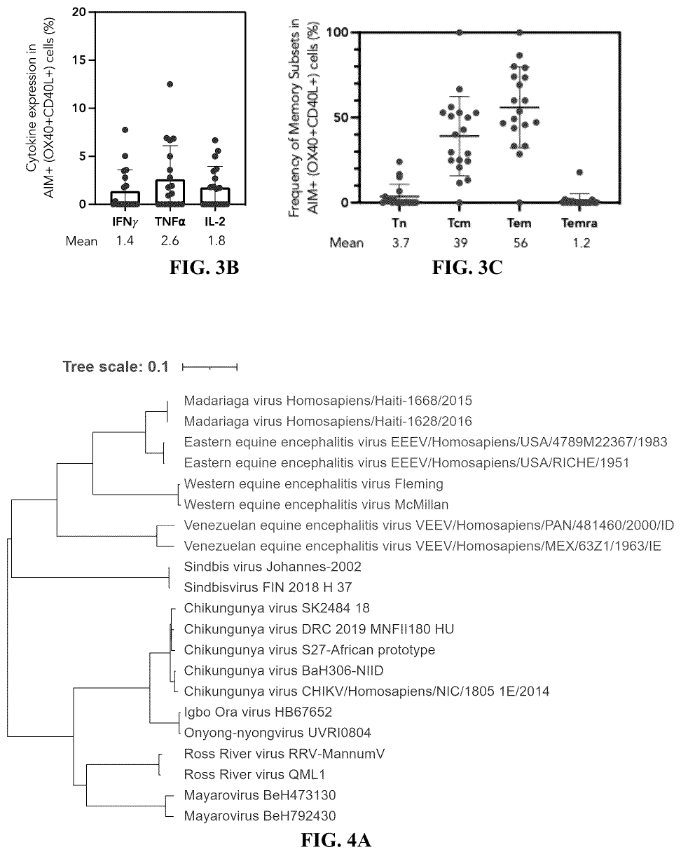

To measure responses to epitope CHIKV MPs, the inventors recruited an independent cohort of 19 CHIKV-seropositive donors with chronic CHIKVD (referred to as chronic CHIKV) and seven CHIKV-seronegative or uninfected controls. Cohort characteristics are provided in Table 4. Infections were confirmed by measuring CHIKV-specific IgG titers (FIG. 6B). The inventors stimulated PBMCs with CHIKV S, CHIKV NS and a combined CHIKV S+NS MPs for 24 h and measured antigen-specific CD4⁺ T cell responses via upregulation of OX40+CD40L+ markers. Post-stimulation with the combined CHIKV S+NS epitope MP, the inventors detected high CHIKV-specific CD4⁺ T cell responses in 9/19 (47%) of donors marked by upregulation of OX40+CD40L+ markers. CHIKV-specific CD4⁺ T cell responses were not detected in uninfected controls as defined by serology (FIG. 3A).

TABLE 4
Characteristics of the CHIKV epitope MP validation cohort.

| Characteristic | Chronic CHIKV | Healthy controls |
|---|---|---|
| Donors, n | 19 | 7 |
| Gender, n (%) | | |
| Male | 6 (32%) | 2 (29%) |
| Female | 13 (68%) | 5 (71%) |
| Age *, years (mean ± SD) | 43.9 ± 15.9 | 43.9 ± 11.7 |
| Years post-infection (mean ± SD) | 6.3 ± 0.4 | NA |
| Current symptoms | | |
| Arthralgia** (%) | 100 | 0 |

* At the time of sample collection
**Includes pain in joints and upper and lower limbs, swelling in limbs, and muscle pain

Next, within the AIM+ specific responses, the inventors measured cytokine expression of IFNγ, TNFα and IL-2 after simulation with the combined CHIKV S+NS MP in chronic CHIKV-seropositive donors via the intracellular staining assay. The inventors detected high levels of TNFα and IL-2 producing CHIKV-specific AIM+ (OX40+CD40L+) CD4⁺ T cells (2.6% for TNFα and 1.8% for IL-2) with lower levels of IFNγ producing CHIKV-specific AIM+ (OX40+CD40L+) CD4⁺ T cells (1.4% for IFNγ) (FIG. 3B). The majority of CHIKV S+NS-specific AIM+ CD4⁺ T cells displayed T effector memory phenotype (Tem: CD45RA-CCR7+), followed by T central memory cell phenotype (Tcm: CD45RA−CCR7−) (FIG. 3C).

Sequence conservation of CHIKV CD4⁺ T cell epitopes in arthritogenic and encephalitic alphaviruses. Targets of CD4⁺ T cell responses in other alphaviruses, including other arthritogenic and encephalitic alphaviruses, are unknown. As such, the inventors wanted to understand to what degree cHIKV epitopes identified in this study are conserved across other arthritogenic and encephalitic alphaviruses. The inventors selected a representative set of viruses from alphavirus genus (phylogeny shown in FIG. 4A) and extracted protein sequences. A calculated percentage of conservation of each CHIKV peptide was calculated using Conservation Analysis tool in IEDB. Plotting the median of percent conservation for arthritogenic (excluding CHIKV sequences; n=8) and encephalitic alphaviruses (n=8) for all CHIKV peptides revealed regions of high conservation, such as in CP, nsP4, nsP1 and nsP2 proteins, and regions of low conservation, such as in 6K, E2 and E1 proteins, in both arthritogenic and encephalitic groups (FIG. 4B). As expected, degree of conservation of CHIKV peptides was higher for all proteins in arthritogenic alphaviruses as opposed to encephalitic alphaviruses. Interestingly, nsP4 protein showed the second highest degree of conservation in both arthritogenic and encephalic groups, however, few epitopes were identified in that region.

For the 123 individual CHIKV epitopes and 24 variant epitopes identified in this study, the inventors plotted the median of percent conservation in arthritogenic and encephalitic alphaviruses (FIG. 4C). Overall, the median of percent conservation for both non-structural and structural epitopes was higher in the arthritogenic group (non-structural: 73%, structural: 67%) as opposed to in the encephalitic group (non-structural: 65%; structural: 47%).

Previous studies have experimentally defined 67% conservation to be associated with cross-reactivity between SARS-CoV-2 epitopes and common-cold coronaviruses [26,27]. As such, the inventors used 67% median conservation as the threshold to identify CHIKV CD4⁺ T cell epitopes that could potentially be cross-reactive with other alphaviruses. Overall, 68% (42/62) of all CHIKV non-structural and 47% (35/75) of structural epitopes were predicted to be potentially cross-reactive with other arthritogenic viruses and 31% (19/62) of the CHIKV non-structural and 8% (10/75) of the structural epitopes were predicted to be potentially cross-reactive with other encephalitic viruses.

A further analysis was focused on epitopes that were recognized in two or more donors (referred to as immunogenic). Five epitopes in the non-structural proteins and 35 epitopes in the structural proteins were defined to be immunogenic. Of the four immunogenic epitopes in the non-structural protein, 90% (4/5) epitopes and 25% (1/4) of the epitopes were predicted to be potentially cross-reactive with other viruses in arthritogenic and in the encephalitic group, respectively. Meanwhile, out of the 35 immunogenic epitopes in the structural protein, 37% (13/35) and 9% (3/35) of the epitopes were predicted to be cross-reactive in the arthritogenic and encephalitic groups, respectively. This potential cross-reactivity between other representatives of the alphavirus genus could be important in the context of infection or vaccination with closely related alphaviruses.

The inventors investigated the repertoire of CHIKV-specific CD4⁺ T cell responses in individuals with chronic CHIKVD. To the best of the inventors' knowledge, no studies so far have reported CHIKV targets of CD4⁺ T cells in humans. The inventors identified 123 novel CHIKV epitopes, 30 of which were recognized by two or more donors. Epitopes in E1 and nsP1 proteins nearly accounted for half of the identified epitopes and more than half of the total magnitude of the response. Specifically, two regions in nsP1 (226-235aa) and E1 (1126-1140aa) proteins were identified as immunodominant, with the highest response frequency and high magnitude of response compared to other CHIKV proteins. CP contained the next highest number of epitopes, which were also frequently recognized and elicited a high magnitude of response. Overall, nsP1, E1 and CP proteins were the most immunogenic, highlighting their importance for vaccine development. In addition, only four epitopes in non-structural proteins were immunogenic as compared to 35 epitopes in the structural protein, suggesting structural proteins to be more immunogenic.

While no CHIKV-specific T cell epitopes have been identified in humans, a few studies have analyzed CHIKV-specific T cell targets in mice. Interestingly, nine of the 26 epitopes defined in CHIKV-infected C5BL/6 mice overlap with the epitopes in the study in humans, which is remarkable given the highly distinct MHC in humans and mice [21]. Specific sequences of the two pathogenic CD4⁺ T cell targets discovered in mice by Teo et al. were found to be immunogenic in this study as well [19]. This suggests that murine models can be a representative model to study CHIKV T cell responses relevant for human disease.

Understanding conservation of T cell targets of CHIKV and other alphaviruses is essential to develop effective universal alphaviral vaccines. This analysis revealed regions of broad conservation of CHIKV proteome in arthritogenic and encephalitic alphaviruses. nsP4 protein had 84% median conservation in arthritogenic alphaviruses, although only three epitopes were identified in nsP4. On the other hand, structural proteins, specifically CP, E2 and E1 proteins, elicited a high magnitude of response with a high number of immunogenic epitopes, however, the percent of conservation was lower than 67%, indicating lower potential cross-reactivity with other alphaviruses. A universal alphaviral vaccine will capture regions of the viral proteome with higher number of T cell epitopes and percent conservation, such as those described herein.

This example provides an in-depth characterization of CHIKV CD4⁺ T cell epitopes in chronic CHIKVD and extends the analysis to determine conservation of the identified epitopes in arthritogenic and encephalitic alphaviruses. Additionally, a CHIKV-specific CD4 epitope MP was designed to characterize CD4⁺ T cell response with smaller blood volumes. These epitopes can be used for an alphaviral vaccine.

TABLE 5
Antibodies used in AIM assay.

| Antibody | Clone (Vendor) | Catalog no. |
|---|---|---|
| CD40 | HB14 (Miltenyi Biotec) | 130-108-041 |
| CCR6-BUV496 | 11A9 (BD Biosciences) | 612948 |
| CXCR5-BV421 | J252D4 (BioLegend) | 356920 |
| CXCR3-BV605 | G025H7 (BioLegend) | 353728 |
| CCR7-BV711 | G043H7 (BioLegend) | 353228 |
| LIVE/DEAD-Fixable Blue | (ThermoFisher) | L23105 |
| CD3-BUV395 | UCHT1 (BD Biosciences) | 563546 |
| CD8-BUV805 | SK1 (BD Biosciences) | 612889 |
| CD16-BV510 | 3G8 (BioLegend) | 302048 |
| CD14-BV510 | 63D3 (BioLegend) | 367124 |
| CD20-BV510 | 2H7 (BioLegend) | 302340 |
| CD45RA-BV570 | Hl100 (BioLegend) | 304132 |
| CD4-cFluor b548 | SK3 (Cytek) | R7-20043 |
| CD95-BB700 | DX2 (BD Biosciences) | 566542 |
| HLA-DR-APC-R700 | G46-6 (BD Biosciences) | 565127 |
| CD38-BV650 | HB-7 (BioLegend) | 356620 |
| PD-1-BV785 | EH12.2H7 (BioLegend) | 329930 |
| CD40L-PE-Dazzle594 | 24-31 (BioLegend) | 310840 |
| OX40-APC | Ber-Act35 (BioLegend) | 350008 |
| CD69-FITC | FN50 (BioLegend) | 310904 |
| CD137-BUV737 | 4b4-1 (BD Biosciences) | 741861 |

TABLE 6
Antibodies used in AIM/AICS assay.

| Antibody | Clone (Source) | Catalog no. |
|---|---|---|
| CD40 | HB14 (Miltenyi Biotec) | 130-094-133 |
| CCR6-BUV496 | 11A9 (Biolegend) | 612948 |
| CXCR5-BV421 | J252D4 (Biolegend) | 356920 |
| CXCR3-BV605 | G025H7 (Biolegend) | 353728 |
| CCR7-BV711 | G043H7 (Biolegend) | 353228 |
| CCR4-APC | L291H4 (Biolegend) | 359408 |
| CD69 - PE | FN50 (BD Biosciences) | 555531 |
| CD137 - PE-Cy5 | 4B4-1 (BD Biosciences) | 551137 |
| LIVE/DEAD-Fixable Blue | (ThermoFisher) | L23105 |
| CD3-BUV395 | UCHT1 (BD Biosciences) | 563546 |
| CD8-BUV805 | SK1 (BD Biosciences) | 612889 |
| CD16-BV510 | 3G8 (Biolegend) | 302048 |
| CD14-BV510 | 63D3 (Biolegend) | 367124 |
| CD20-BV510 | 2H7 (Biolegend) | 302340 |
| CD45RA-BV570 | HI100 (Biolegend) | 304132 |
| CD4-cFluor b548 | SK3 (Cytek) | R7-20043 |
| HLA-DR-APC R700 | G46-6 (BD Biosciences) | 565127 |
| CD38-BV650 | HB-7 (Biolegend) | 353228 |
| PD-1-BV480 | EH12.1 (BD Biosciences) | 566112 |
| OX40-PE-Cy7 | Ber-ACT35 (Biolegend) | 350012 |
| IFNg-FITC | 4S.B3 (eBioscience) | 11-7319-82 |
| IL-17-BV785 | BL168 (Biolegend) | 512338 |
| IL-10-PE-Dazzle594 | JES3-19F1 (Biolegend) | 506812 |
| IL-2-BUV737 | (BD Biosciences) | 612836 |
| TNFa-eFluor450 | Mab11 (eBiosciences) | 48-7349-42 |
| Granzyme B-AF647 | GR11 (BD Biosciences) | 560212 |
| CD40L-APC-efluor 780 | 24-31 (ThermoFisher) | 46-1548-42 |

Mechanism of immunodominance for the nsP1₂₂₆₋₂₃₅, E1₁₁₂₆₋₁₁₄₀ and CP₄₆₋₅₀ regions.

The data presented above pin-points residues nsP1₂₂₆₋₂₃₅, E1₁₁₂₆₋₁₁₄₀ and CP₄₆₋₅₀ as being associated with remarkable frequency and magnitude of responses. Additional experiment addressed the molecular mechanisms associated with such dominance, and whether these regions would likely contain a single epitope encoded in the overlap between the two consecutive 15mer peptides, or whether they would happen to contain multiple distinct epitopes.

Immunodominant epitopes in CHIKV recognized by CD4⁺ T cells.

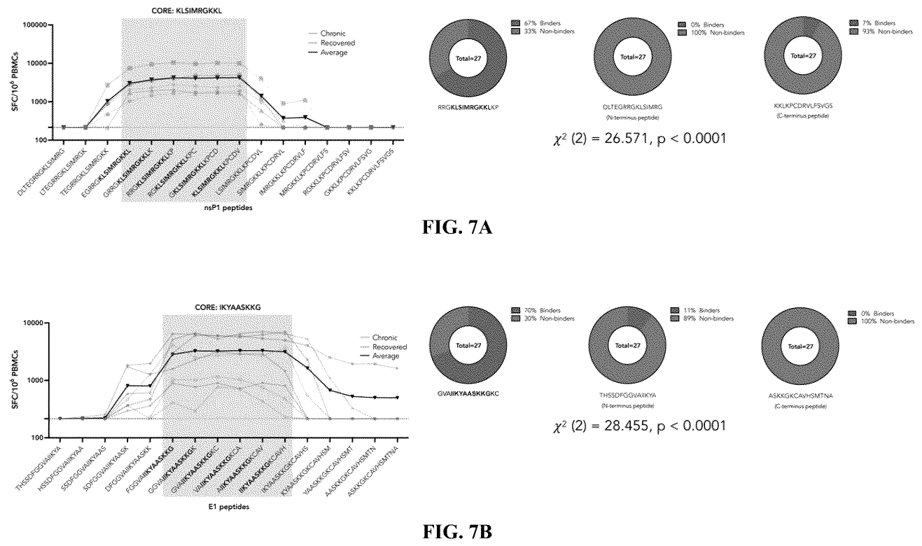

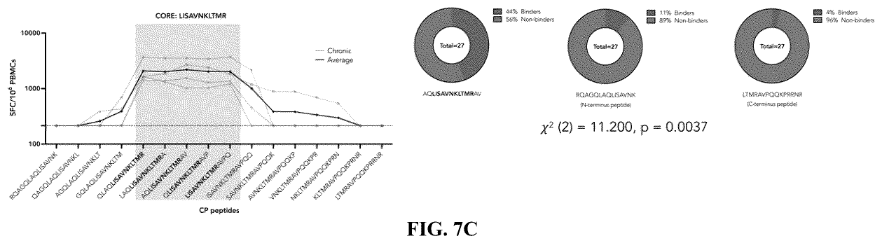

FIGS. 7A to 7C show the immunodominant epitopes in CHIKV recognized by CD4⁺ T cells. (FIG. 7A), (FIG. 7B) and (FIG. 7C) SFC/10⁶ PBMCs shown for individual chronic (blue line) and recovered (gray line) donors against 15mer peptides overlapping by 14 residues sequentially spanning the immunodominant region of (FIG. 7A) the nsP1 (chronic: n=3, recovered: n=2), (FIG. 7B) E1 (chronic: n=5, recovered: n=3) and (FIG. 7C) CP (chronic: n=4) regions. The black line shows the average SFC/10⁶ PBMCs for all donors shown. Blue highlighted regions and bolded peptide sequences indicate region with the highest response and the common amino acid sequence, respectively. The pie charts on the right depict the percentage of HLA alleles predicted to bind to the peptide sequences (i.e. the mapped epitope and negative control peptides on the N- and C-termini) shown under the pie chart. Chi-square value with degree of freedom and p-value is reported for all peptide sequences. Data are represented as mean±SD or geomean±geometric SD.

A series of 15mer peptides were synthetized overlapping by 14 residues and spanning residues 216-245 for nsP1, 1116-1145 for E1, and 36-65 for CP. These peptides were tested for immunogenicity with a T cell line derived by two-week in vitro restimulation of PBMCs from chronic and recovered donors (fine epitope mapping cohort from Table 7). Plotting responses from all donors clearly indicated that the peptides associated with optimal response all share the KLSIMRGKKL (SEQ ID NO: 148), IKYAASKKG (SEQ ID NO: 149) and LISAVNKLTMR (SEQ ID NO: 150) core regions for nsP1 (FIG. 7A), E1 (FIG. 7B) and CP (FIG. 7C), respectively. In addition, T cells from recovered individuals recognized the same core sequences for nsP1 and E1 proteins, but at a considerably lower magnitude as compared to chronic donors.

TABLE 7
Characteristics of the donor cohort.

| | CHIKV | Epitope | | Fine epitope mapping | | | | Epitope MP Validation | | | |
|---|---|---|---|---|---|---|---|---|---|---|---|
| | seronegative | Screening | | cohort | | | | cohort | | | |
| Characteristic | cohort | cohort | | Chronic | | Recovered | | Chronic | | Uninfected | |
| Donors, n | 6 | 17 | | 10 | | 4 | | 19 | | 7 | |
| Gender, n (%) | | | | | | | | | | | |
| Male | NA | 6 | (35%) | 2 | (20%) | 0 | (0%) | 6 | (32%) | 2 | (29%) |
| Female | NA | 11 | (65%) | 8 | (80%) | 4 | (100%) | 13 | (68%) | 5 | (71%) |

| | | | | | | |
|---|---|---|---|---|---|---|
| Age *, years (mean ± SD) | NA | 46.8 ± 14 | 44.1 ± 13 | 34.5 ± 2.6 | 43.9 ± 16 | 43.9 ± 12 |
| Years post-infection (mean ± SD) | | 6.3 ± 0.5 | 6.2 ± 0.6 | 6.4 ± 0.3 | 6.3 ± 0.4 | |
| Current symptoms | | | | | | |
| Arthralgia** (%) | 0 | 100 | 100 | 0 | 100 | 0 |
| CHIKV serostatus | Negative | Positive | Positive | Positive | Positive | Negative |

| | | | | |
|---|---|---|---|---|
| Site | Nicaragua | Colombia | Colombia | Colombia |

* At the time of sample collection
**Includes pain in joints and upper and lower limbs, swelling in limbs, and muscle pain
NA—Information not available

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Suhrbier A, La Linn M. Clinical and pathologic aspects of arthritis due to Ross River virus and other alphaviruses. Current Opinion in Rheumatology. 2004; 16: 374. doi:10.1097/01.bor.0000130537.76808.26.
2. Toivanen A. Alphaviruses: an emerging cause of arthritis? Current Opinion in Rheumatology. 2008; 20: 486. doi:10.1097/BOR.0b013e328303220b.
3. Zacks M A, Paessler S. ENCEPHALITIC ALPHAVIRUSES. Vet Microbiol. 2010; 140: 281. doi:10.1016/j.vetmic.2009.08.023.
4. Powers A M, Brault A C, Shirako Y, Strauss E G, Kang W, Strauss J H, et al. Evolutionary Relationships and Systematics of the Alphaviruses. J Virol. 2001; 75: 10118-10131. doi:10.1128/JVI.75.21.10118-10131.2001.
5. Gérardin P, Barau G, Michault A, Bintner M, Randrianaivo H, Choker G, et al. Multidisciplinary Prospective Study of Mother-to-Child Chikungunya Virus Infections on the Island of La Réunion. PLOS Medicine. 2008; 5: e60. doi:10.1371/journal.pmed.0050060.
6. Borgherini G, Poubeau P, Jossaume A, Gouix A, Cotte L, Michault A, et al. Persistent Arthralgia Associated with Chikungunya Virus: A Study of 88 Adult Patients on Reunion Island. Clinical Infectious Diseases. 2008; 47: 469-475. doi:10.1086/590003.
7. Schilte C, Staikovsky F, Couderc T, Madec Y, Carpentier F, Kassab S, et al. Chikungunya Virus-associated Long-term Arthralgia: A 36-month Prospective Longitudinal Study. PLOS Neglected Tropical Diseases. 2013; 7: e2137. doi:10.1371/journal.pntd.0002137.
8. Borgherini G, Poubeau P, Jossaume A, Gouix A, Cotte L, Michault A, et al. Persistent Arthralgia Associated with Chikungunya Virus: A Study of 88 Adult Patients on Reunion Island. Clinical Infectious Diseases. 2008; 47: 469-475. doi:10.1086/590003.
9. Larrieu S, Pouderoux N, Pistone T, Filleul L, Receveur M-C, Sissoko D, et al. Factors associated with persistence of arthralgia among chikungunya virus-infected travellers: Report of 42 French cases. Journal of Clinical Virology. 2010; 47: 85-88. doi:10.1016/j.jcv.2009.11.014.
10. Couturier E, Guillemin F, Mura M, Léon L, Virion J-M, Letort M-J, et al. Impaired quality of life after chikungunya virus infection: a 2-year follow-up study. Rheumatology. 2012; 51: 1315-1322. doi:10.1093/rheumatology/kes015.
11. Powers A M, Logue C H. Changing patterns of chikungunya virus: re-emergence of a zoonotic arbovirus. Journal of General Virology. 2007; 88: 2363-2377. doi:10.1099/vir.0.82858-0.
12. Chikungunya virus disease cases January-December 2023. 29 Jan. 2024 [cited 29 Jul. 2024]. Available: https://www.ecdc.europa.eu/en/publications-data/chikungunya-virus-disease-cases-january-december-2023.
13. Ryan S J, Carlson C J, Mordecai E A, Johnson L R. Global expansion and redistribution of Aedes-borne virus transmission risk with climate change. PLoS Negl Trop Dis. 2019; 13: e0007213. doi:10.1371/journal.pntd.0007213.
14. Simon F, Savini H, Parola P. Chikungunya: A Paradigm of Emergence and Globalization of Vector-Borne Diseases. Medical Clinics of North America. 2008; 92: 1323-1343. doi:10.1016/j.mcna.2008.07.008.
15. Tan Y B, Chmielewski D, Law M C Y, Zhang K, He Y, Chen M, et al. Molecular architecture of the Chikungunya virus replication complex. Sci Adv. 8: eadd2536. doi:10.1126/sciadv.add2536.
16. Metz S W, Pijlman G P. Function of Chikungunya Virus Structural Proteins. In: Okeoma C M, editor. Chikungunya Virus: Advances in Biology, Pathogenesis, and Treatment. Cham: Springer International Publishing; 2016. pp. 63-74. doi:10.1007/978-3-319-42958-8_5.
17. Teo T-H, Lum F-M, Claser C, Lulla V, Lulla A, Merits A, et al. A Pathogenic Role for CD4+ T Cells during Chikungunya Virus Infection in Mice. The Journal of Immunology. 2013; 190: 259-269. doi:10.4049/jimmunol.1202177.
18. Morrison T E, Oko L, Montgomery S A, Whitmore A C, Lotstein A R, Gunn B M, et al. A Mouse Model of Chikungunya Virus-Induced Musculoskeletal Inflammatory Disease: Evidence of Arthritis, Tenosynovitis, Myositis, and Persistence. The American Journal of Pathology. 2011; 178: 32-40. doi:10.1016/j.ajpath.2010.11.018.
19. Teo T-H, Chan Y-H, Lee W W L, Lum F-M, Amrun S N, Her Z, et al. Fingolimod treatment abrogates chikungunya virus-induced arthralgia. Science Translational Medicine. 2017; 9: eaa11333. doi:10.1126/scitranslmed.aa11333.
20. Chang J, Agarwal R, Côrtes F H, Ha C, Villalpando J, Castillo I N, et al. Chikungunya-virus-specific CD4+ T cells are associated with chronic chikungunya viral arthritic disease in humans. bioRxiv; 2023. p. 2023.11.20.567688. doi:10.1101/2023.11.20.567688.
21. Broeckel R M, Haese N, Ando T, Dmitriev I, Kreklywich C N, Powers J, et al. Vaccine-Induced Skewing of T Cell Responses Protects Against Chikungunya Virus Disease. Front Immunol. 2019; 10: 2563. doi:10.3389/fimmu.2019.02563.
22. Waheed Y, Safi S Z, Najmi M H, Aziz H, Imran M. Prediction of promiscuous T cell epitopes in RNA dependent RNA polymerase of Chikungunya virus. Asian Pacific Journal of Tropical Medicine. 2017; 10: 760-764. doi:10.1016/j.apjtm.2017.07.023.
23. Pratheek B M, Suryawanshi A R, Chattopadhyay S, Chattopadhyay S. In silico analysis of MHC-I restricted epitopes of Chikungunya virus proteins: Implication in understanding anti-CHIKV CD8+ T cell response and advancement of epitope based immunotherapy for CHIKV infection. Infection, Genetics and Evolution. 2015; 31: 118-126. doi:10.1016/j.meegid.2015.01.017.
24. Sánchez-Burgos G G, Montalvo-Marin N M, Díaz-Rosado E R, Pérez-Rueda E. In Silico Identification of Chikungunya Virus B- and T-Cell Epitopes with High Antigenic Potential for Vaccine Development. Viruses. 2021; 13: 2360. doi:10.3390/v13122360.
25. da Silva Antunes R, Weiskopf D, Sidney J, Rubiro P, Peters B, Lindestam Arlehamn C S, et al. The MegaPool Approach to Characterize Adaptive CD4+ and CD8+ T Cell Responses. Current Protocols. 2023; 3: e934. doi:10.1002/cpz1.934.
26. Mateus J, Grifoni A, Tarke A, Sidney J, Ramirez S I, Dan J M, et al. Selective and cross-reactive SARS-CoV-2 T cell epitopes in unexposed humans. Science. 2020; 370: 89-94. doi:10.1126/science.abd3871.
27. Tarke A, Zhang Y, Methot N, Narowski T M, Phillips E, Mallal S, et al. Targets and cross-reactivity of human T cell recognition of common cold coronaviruses. Cell Reports Medicine. 2023; 4: 101088. doi:10.1016/j.xcrm.2023.101088.
28. Lemieux A, Sannier G, Nicolas A, Nayrac M, Delgado G-G, Cloutier R, et al. Enhanced detection of antigen-specific T cells by a multiplexed AIM assay. Cell Rep Methods. 2024; 4: 100690. doi:10.1016/j.crmeth.2023.100690.
29. Grifoni A, Weiskopf D, Ramirez S I, Mateus J, Dan J M, Moderbacher C R, et al. Targets of T Cell Responses to SARS-CoV-2 Coronavirus in Humans with COVID-19 Disease and Unexposed Individuals. Cell. 2020; 181: 1489-1501.e15. doi:10.1016/j.cell.2020.05.015.
30. Hinz D, Seumois G, Gholami A M, Greenbaum J A, Lane J, White B, et al. Lack of allergy to timothy grass pollen is not a passive phenomenon but associated with allergen-specific modulation of immune reactivity. Clin Exp Allergy. 2016; 46: 705-719. doi:10.1111/cea.12692.
31. Lindestam Arlehamn C S, McKinney D M, Carpenter C, Paul S, Rozot V, Makgotlho E, et al. A Quantitative Analysis of Complexity of Human Pathogen-Specific CD4 T Cell Responses in Healthy M. tuberculosis Infected South Africans. PLoS Pathog. 2016; 12: e1005760. doi:10.1371/journal.ppat.1005760.
32. Bancroft T, Dillon MBC, Antunes R da S, Paul S, Peters B, Crotty S, et al. Th1 versus Th2 T cell polarization by whole-cell and acellular childhood pertussis vaccines persists upon re-immunization in adolescence and adulthood. Cell Immunol. 2016; 304-305: 35-43. doi:10.1016/j.cellimm.2016.05.002.
33. da Silva Antunes R, Paul S, Sidney J, Weiskopf D, Dan J M, Phillips E, et al. Definition of Human Epitopes Recognized in Tetanus Toxoid and Development of an Assay Strategy to Detect Ex Vivo Tetanus CD4+ T Cell Responses. PLoS One. 2017; 12: e0169086. doi:10.1371/journal.pone.0169086.
34. Grifoni A, Angelo M A, Lopez B, O'Rourke P H, Sidney J, Cerpas C, et al. Global Assessment of Dengue Virus-Specific CD4+ T Cell Responses in Dengue-Endemic Areas. Front Immunol. 2017; 8: 1309. doi:10.3389/fimmu.2017.01309.
35. Weiskopf D, Cerpas C, Angelo M A, Bangs D J, Sidney J, Paul S, et al. Human CD8+ T-Cell Responses Against the 4 Dengue Virus Serotypes Are Associated With Distinct Patterns of Protein Targets. J Infect Dis. 2015; 212: 1743-1751. doi:10.1093/infdis/jiv289.
36. Weiskopf D, Angelo M A, de Azeredo E L, Sidney J, Greenbaum J A, Fernando A N, et al. Comprehensive analysis of dengue virus-specific responses supports an HLA-linked protective role for CD8+ T cells. Proc Natl Acad Sci USA. 2013; 110: E2046-E2053. doi:10.1073/pnas.1305227110.
37. Weiskopf D, Bangs D J, Sidney J, Kolla R V, De Silva A D, de Silva A M, et al. Dengue virus infection elicits highly polarized CX3CR1+ cytotoxic CD4+ T cells associated with protective immunity. Proc Natl Acad Sci USA. 2015; 112: E4256-E4263. doi:10.1073/pnas.1505956112
38. Dahora L, Castillo I N, Medina F A, Vila F, Munoz-Jordan J, Segovia B, et al. Flavivirus Serologic Surveillance: Multiplex Sample-Sparing Assay for Detecting Type-Specific Antibodies to Zika and Dengue Viruses. Rochester, NY; 2023. doi:10.2139/ssrn.4411430.

What is claimed is:

1. A composition comprising:
one or more peptides or proteins, comprising, consisting of, or consisting essentially of an amino acid sequence selected from any sequence set forth in Table 1 (SEQ ID NOS: 1 to 150), or a subsequence, portion, homologue, variant or derivative thereof;
a fusion protein comprising one or more amino acid sequences selected from any sequence set forth in Table 1 (SEQ ID NOS: 1 to 150); or
a pool of 2 or more or more peptides comprising, consisting of, or consisting essentially of amino acid sequences selected from any sequence set forth in Table 1 (SEQ ID NOS: 1 to 150); or
a polynucleotide that encodes one or more peptides or proteins, comprising, consisting of, or consisting essentially of an amino acid sequence selected from any sequence set forth in Table 1 (SEQ ID NOS: 1 to 150), or a subsequence, portion, homologue, variant or derivative thereof.

2. The composition of claim 1, wherein the one or more peptides or proteins comprises, or wherein the fusion protein comprises 2 or more or more amino acid sequences selected from any sequence set forth in Table 1 (SEQ ID NOS: 1 to 150), or a subsequence, portion, homologue, variant or derivative thereof;
the amino acid sequence is selected from an Alphavirus T cell epitope selected from any sequence set forth in Table 1 (SEQ ID NOS: 1 to 150);
the composition comprises one or more Chikungunya virus peptides amino acid sequences selected from any sequence set forth in Table 1 (SEQ ID NOS: 1 to 150), or a subsequence, portion, homologue, variant or derivative thereof;
a fusion protein comprising one or more amino acid sequences selected from any sequence set forth in Table 1 (SEQ ID NOS: 1 to 150); or
a pool of 2 or more peptides selected from any sequence set forth in Table 1 (SEQ ID NOS: 1 to 150); or
a polynucleotide that encodes one or more peptides or proteins, comprising, consisting of, or consisting essentially of an amino acid sequence selected from any sequence set forth in Table 1 (SEQ ID NOS: 1 to 150), or a subsequence, portion, homologue, variant or derivative thereof;
the peptide or protein comprises an Alphavirus T cell epitope;
the one or more peptides or proteins comprises an Alphavirus CD8+ or CD4+ T cell epitope;
the Alphavirus is Chikungunya virus and the Chikungunya virus T cell epitope is not conserved in another Alphavirus;
the Alphavirus is Chikungunya virus and the Chikungunya virus T cell epitope is conserved in another Alphavirus;
one or more peptides or proteins has a length from about 9-15, 15-20, 20-25, 25-30, 30-40, 40-50, 50-75 or 75-100 amino acids;
one or more peptides or proteins elicits, stimulates, induces, promotes, increases or enhances a T cell response to an Alphavirus;
one or more peptides or proteins that elicits, stimulates, induces, promotes, increases or enhances the T cell response to the Alphavirus is an Alphavirus protein or peptide, or a variant, homologue, derivative or subsequence thereof.

3. The composition of claim 1, further comprising formulating the one or more peptides or proteins into an immunogenic formulation with an adjuvant, and the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands;
a modulator of immune response;
a modulator of the innate immune response, wherein the modulator is Interleukin-6 (IL-6), Interferon-gamma (IFN-γ), Transforming growth factor beta (TGF-β), or Interleukin-10 (IL-10), or an agonist or antagonist thereof.

4. The composition of claim 1, wherein the composition comprises monomers or multimers of:
peptides or proteins comprising, consisting of, or consisting essentially of:
one or more amino acid sequences selected from any sequence set forth in Table 1 (SEQ ID NOS: 1 to 150),
concatemers, subsequences, portions, homologues, variants or derivatives thereof;
a fusion protein comprising one or more amino acid sequences selected from any sequence set forth in Table 1 (SEQ ID NOS: 1 to 150); or
a polynucleotide that encodes one or more peptides or proteins, comprising, consisting of, or consisting essentially of an amino acid sequence selected from any sequence set forth in Table 1 (SEQ ID NOS: 1 to 150), or a subsequence, portion, homologue, variant or derivative thereof.

5. The composition of claim 1, further comprising one or more peptide-major histocompatibility complex (MHC) monomers or multimers, wherein a peptide-MHC monomer or multimer comprises a peptide comprising, consisting of, or consisting essentially of an amino acid sequence selected from any sequence set forth in Table 1 (SEQ ID NOS: 1 to 150), in a groove of the MHC monomer or multimer.

6. A method for detecting the presence of: (i) an Alphavirus or (ii) an immune response relevant to Alphavirus infections, vaccines or therapies, including T cells responsive to one or more Alphavirus peptides, comprising:
providing one or more proteins or peptides for detection of an amount or a relative amount of, and/or the activity of, and/or the state of antigen-specific T-cells;
contacting a biological sample suspected of having Alphavirus-specific T-cells to one or more proteins or peptides for detection; and
detecting an amount or a relative amount of, and/or the activity of, and/or the state of antigen-specific T-cells in the biological sample, wherein the one or more proteins or peptides for detection comprise one or more amino acid sequences set forth in Table 1 (SEQ ID NOS: 1 to 150), or comprise a pool of 2 or more or more amino acid sequences set forth in Table 1 (SEQ ID NOS: 1 to 150).

7. The method of claim 6, further comprising at least one of:
detecting an amount or a relative amount of, and/or activity of antigen-specific T-cells comprises one or more steps of identification or detection of the antigen-specific T-cells and measuring the amount of the antigen-specific T-cells;
detecting an amount or a relative amount of, and/or activity of antigen-specific T-cells comprises indirect detection and/or direct detection;
detecting an amount or a relative amount of, and/or the activity of, and/or an activation state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay; or
detecting the presence or amount of the one or more peptides in a biological sample, or a response thereto, which is diagnostic of an Alphavirus infection.

8. The method of claim 6, wherein the one or more peptides or proteins comprises 2 or more amino acid sequences selected from those set forth in Table 1 (SEQ ID NOS: 1 to 150).

9. The method of claim 6, wherein the method of detecting an immune response relevant to the Alphavirus comprises the following steps:
providing an MHC monomer or an MHC multimer;
contacting a population T-cells to the MHC monomer or MHC multimer; and
measuring the number, activity or state of T-cells specific for the MHC monomer or MHC multimer; or
wherein the MHC monomer or MHC multimer comprises a protein or peptide of the Alphavirus.

10. The method of claim 6, wherein at least one of:
the protein or peptide comprises a CD8+ or CD4+ T cell epitope;
the T cell epitope is not conserved in another Alphavirus;
the T cell epitope is conserved in another Alphavirus;
the protein or peptide has a length from about 9-15, 15-20, 20-25, 25-30, 30-40, 40-50, 50-75 or 75-100 amino acids;
the proteins or peptides comprise 2 or more amino acid sequences selected from any sequence set forth in Table 1 (SEQ ID NOS: 1 to 150), or a subsequence, portion, homologue, variant or derivative thereof.

11. The method of claim 6, further comprising administering a treatment comprising the composition to the subject from which the biological sample was drawn that increases the amount or relative amount of, and/or activity of the antigen-specific T-cells.

12. The method of claim 6, further comprising detecting the presence of: (i) Chikungunya virus or (ii) an immune response relevant to Chikungunya virus infections, vaccines or therapies, including T cells responsive to one or more Chikungunya virus peptides, comprising:
providing one or more proteins or peptides for detection of an amount or a relative amount of, and/or the activity of, and/or the state of antigen-specific T-cells;
contacting a biological sample suspected of having Chikungunya virus-specific T-cells to one or more proteins or peptides for detection; and
detecting an amount or a relative amount of, and/or the activity of, and/or the state of antigen-specific T-cells in the biological sample, wherein the one or more proteins or peptides for detection comprise one or more amino acid sequences set forth in those sequences set forth in Table 1 (SEQ ID NOS: 1 to 150), or comprise a pool of 2 or more amino acid sequences set forth in those sequences set forth in Table 1 (SEQ ID NOS: 1 to 150).

13. The method of claim 6, further comprising
contacting a biological sample from a subject with a composition; and
determining if the composition elicits an immune response from the contacted cells, wherein the presence of an immune response indicates that the subject has been exposed to or infected with Alphavirus, wherein:
the sample comprises T cells;
the response comprises inducing, increasing, promoting or stimulating anti-Alphavirus activity of T cells; or
the T cells are CD8+ or CD4+ T cells.

14. The method of claim 6, wherein the method comprises at least one of:
determining whether the subject has been infected by or exposed to the Alphavirus more than once by determining if the subject elicits a secondary T cell immune response profile that is different from a primary T cell immune response profile; or
diagnosing an Alphavirus infection or exposure in a subject, the method comprising contacting a biological sample from a subject with a composition comprising the one or more proteins or peptides, and determining if the composition elicits a T cell immune response, wherein the T cell immune response identifies that the subject has been infected with or exposed to an Alphavirus,
wherein the method is conducted three or more days following the date of suspected infection by or exposure to an Alphavirus.

15. A kit for the detection of Alphavirus or an immune response to Alphavirus in a subject comprising, consisting of or consisting essentially of:
one or more T cells that specifically detect the presence of:
one or more amino acid sequences selected from any sequence set forth in Table 1 (SEQ ID NOS: 1 to 150), or a subsequence, portion, homologue, variant or derivative thereof; or
a fusion protein comprising one or more amino acid sequences selected from any sequence set forth in Table 1 (SEQ ID NOS: 1 to 150); or
a pool of 2 or more or more peptides selected from the amino acid sequences set forth in Table 1 (SEQ ID NOS: 1 to 150).

16. The kit of claim 15, wherein at least one of:
the one or more amino acid sequences are selected from an Alphavirus T cell epitope set forth in any one of Table 1 (SEQ ID NOS: 1 to 150);
the composition comprises: one or more amino acid sequences selected from any sequence set forth in Table 1 (SEQ ID NOS: 1 to 150), or a subsequence, portion, homologue, variant or derivative thereof, a fusion protein comprising one or more amino acid sequences selected from any sequence set forth in Table 1 (SEQ ID NOS: 1 to 150); or a pool of 2 or more peptides selected from the amino acid sequences set forth in those sequences set forth in Table 1 (SEQ ID NOS: 1 to 150);
the amino acid sequence comprises an Alphavirus CD8+ or CD4+ T cell epitope;
the T cell epitope is not conserved in another Alphavirus;
the T cell epitope is conserved in another Alphavirus;
the fusion protein has a length from about 9-15, 15-20, 20-25, 25-30, 30-40, 40-50, 50-75 or 75-100 amino acids;
the kit includes instruction for a diagnostic method, a process, a composition, a product, a service or component part thereof for the detection of: (i) Alphavirus or (ii) an immune response relevant to Alphavirus infections, vaccines or therapies, including T cells responsive to Alphavirus;
the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay; or
the kit includes reagents for determining a Human Leukocyte Antigen (HLA) profile of a subject, and selecting peptides that are presented by the HLA profile of the subject for detecting an immune response to Alphavirus.

17. The kit of claim 15, wherein the Alphavirus is a Chikungunya virus or an immune response to Chikungunya virus in a subject comprising, consisting of or consisting essentially of:
one or more T cells that specifically detect the presence of:
one or more amino acid sequences selected from any sequence set forth in Table 1 (SEQ ID NOS: 1 to 150), or a subsequence, portion, homologue, variant or derivative thereof;
a fusion protein comprising one or more amino acid sequences selected from any sequence set forth in Table 1 (SEQ ID NOS: 1 to 150); or
a pool of 2 or more peptides selected from the amino acid sequences set forth in those sequences set forth in Table 1 (SEQ ID NOS: 1 to 150).

18. A method of stimulating, inducing, promoting, increasing, or enhancing an immune response against an Alphavirus in a subject, comprising:
administering a composition comprising:
one or more peptides or proteins, comprising, consisting of, or consisting essentially of an amino acid sequence selected from any sequence set forth in Table 1 (SEQ ID NOS: 1 to 150), or a subsequence, portion, homologue, variant or derivative thereof;
a fusion protein comprising one or more amino acid sequences selected from any sequence set forth in Table 1 (SEQ ID NOS: 1 to 150); or
a pool of 2 or more or more peptides comprising, consisting of, or consisting essentially of amino acid sequences selected from any sequence set forth in Table 1 (SEQ ID NOS: 1 to 150); or
a polynucleotide that encodes one or more peptides or proteins, comprising, consisting of, or consisting essentially of an amino acid sequence selected from any sequence set forth in Table 1 (SEQ ID NOS: 1 to 150), or a subsequence, portion, homologue, variant or derivative thereof, in an amount sufficient to stimulate, induce, promote, increase, or enhance an immune response against the Alphavirus in the subject.

19. The method of claim 18, wherein the immune response provides the subject with protection against an Alphavirus infection or pathology, or one or more physiological conditions, disorders, illnesses, diseases or symptoms caused by or associated with Alphavirus infection or pathology; or
the immune response is specific to: one or more Chikungunya virus peptides selected from the amino acid sequences set forth in Table 1 (SEQ ID NOS: 1 to 150), or a subsequence, portion, homologue, variant or derivative thereof, or both.

20. The method of claim 18, wherein the method of stimulating, inducing, promoting, increasing, or enhancing an immune response against Chikungunya virus in a subject, comprising:
administering to a subject an amount of a protein or peptide or a polynucleotide that expresses the protein or peptide comprising, consisting of or consisting essentially of an amino acid sequence of the Chikungunya viral protein or peptide, or a variant, homologue, derivative or subsequence thereof, wherein the protein or peptide comprises at least two peptides selected from the amino acid sequences set forth in Table 1 (SEQ ID NOS: 1 to 150) or a subsequence, portion, homologue, variant or derivative thereof, in an amount sufficient to prevent, stimulate, induce, promote, increase, immunize against, or enhance an immune response against Chikungunya virus in the subject, wherein the immune response provides the subject with protection against Chikungunya virus infection or pathology, or one or more physiological conditions, disorders, illnesses, diseases or symptoms caused by or associated with Chikungunya virus infection or pathology.

21. The method of claim 20, wherein treating, preventing, or immunizing a subject against Chikungunya virus infection, comprising administering to a subject an amount of a protein, peptide or a polynucleotide that expresses the protein or peptide comprising, consisting of, or consisting essentially of an amino acid sequence of an Alphavirus protein or peptide, or a variant, homologue, derivative or subsequence thereof, wherein the protein or peptide comprises at least two amino acid sequences selected from any one of Table 1 (SEQ ID NOS: 1 to 150) or a subsequence, portion, homologue, variant or derivative thereof, in an amount sufficient to treat, prevent, or immunize the subject for Chikungunya virus infection, wherein the protein or peptide comprises or consists of an Alphavirus T cell epitope that elicits, stimulates, induces, promotes, increases, or enhances an anti-Chikungunya virus T cell immune response.

22. The method of claim 21, wherein at least one of:
the anti-Chikungunya virus T cell response is a CD8+, a CD4+ T cell response, or both;
the T cell epitope is conserved across two or more clinical isolates of Chikungunya virus or two or more circulating forms of Chikungunya virus;
the Chikungunya virus infection is an acute infection;
the subject is a mammal or a human;
the method reduces Chikungunya viral titer, increases or stimulates Chikungunya viral clearance, reduces or inhibits Chikungunya viral proliferation, reduces or inhibits increases in Chikungunya viral titer or Chikungunya viral proliferation, reduces the amount of a Chikungunya viral protein or the amount of a Chikungunya viral nucleic acid, or reduces or inhibits synthesis of a Chikungunya viral protein or a Chikungunya viral nucleic acid;
the method reduces one or more adverse physiological conditions, disorders, illness, diseases, symptoms or complications caused by or associated with Chikungunya virus infection or pathology;
the method improves one or more adverse physiological conditions, disorders, illness, diseases, symptoms or complications caused by or associated with Chikungunya virus infection or pathology;
the symptom is fever or chills, joint pain, fatigue, muscle or body aches, headache, nausea or vomiting, diarrhea, conjunctivitis or rash;
the method reduces or inhibits susceptibility to Chikungunya virus infection or pathology;
the protein or peptide, or a subsequence, portion, homologue, variant or derivative thereof, is administered prior to, substantially contemporaneously with or following exposure to or infection of the subject with Chikungunya virus;
a plurality of Chikungunya virus T cell epitopes are administered prior to, substantially contemporaneously with or following exposure to or infection of the subject with Chikungunya virus;
the protein or peptide, or a subsequence, portion, homologue, variant or derivative thereof is administered within 2-72 hours, 2-48 hours, 4-24 hours, 4-18 hours, or 6-12 hours after a symptom of Chikungunya virus infection or exposure develops;
the protein or peptide, or a subsequence, portion, homologue, variant or derivative thereof is administered prior to exposure to or infection of the subject with Chikungunya virus;
the protein or peptide, or a subsequence, portion, homologue, variant or derivative thereof is provided in an amount sufficient to treat, prevent, or immunize the subject for Chikungunya virus infection.

23. The method of claim 18, wherein the method further comprises administering a modulator of immune response prior to, substantially contemporaneously with or following the administration to the subject of an amount of a protein or peptide;
the modulator of immune response is a modulator of the innate immune response; or
the modulator is IL-6, IFN-γ, TGF-β, or IL-10, or an agonist or antagonist thereof.

24. The method of claim 18, wherein the peptide or peptides that are immunoprevalent or immunodominant in a virus are obtained by a method consisting of, or consisting essentially of:
obtaining an amino acid sequence of the virus;
determining one or more sets of overlapping peptides spanning one or more viral antigen using unbiased selection;
synthesizing one or more pools of viral peptides comprising the one or more sets of overlapping peptides;
combining the one or more pools of viral peptides with Class I major histocompatibility proteins (MHC), Class II MHC, or both Class I and Class II MHC to form peptide-MHC complexes;
contacting the peptide-MHC complexes with T cells from subjects exposed to the viral;
determining which pools triggered cytokine release by the T cells; and
deconvoluting from the pool of peptides that elicited cytokine release by the T cells, which peptide or peptides are immunoprevalent or immunodominant in the pool.

25. A polynucleotide that expresses one or more peptides or proteins, comprising, consisting of, or consisting essentially of an amino acid sequence selected from any sequence set forth in Table 1 (SEQ ID NOS: 1 to 150), or a subsequence, portion, homologue, variant or derivative thereof;
a fusion protein comprising one or more amino acid sequences selected from any sequence set forth in Table 1 (SEQ ID NOS: 1 to 150); or
a pool of 2 or more or more peptides comprising, consisting of, or consisting essentially of amino acid sequences selected from any sequence set forth in Table 1 (SEQ ID NOS: 1 to 150).

26. The polynucleotide of claim 25, further comprising a vector.

27. The polynucleotide of claim 26, further comprising a host cell that comprises the vector.


SEQUENCE LISTING (150 sequences)

SEQ ID NO: 1 - protein, 15 aa, Chikungunya virus
MEFIPTQTFYNRRYQ

SEQ ID NO: 2 - protein, 15 aa, Chikungunya virus
TQTFYNRRYQPRPWT

SEQ ID NO: 3 - protein, 15 aa, Chikungunya virus
NRRYQPRPWTPRPTI

SEQ ID NO: 4 - protein, 15 aa, Chikungunya virus
PRPWTPRPTIQVIRP

SEQ ID NO: 5 - protein, 15 aa, Chikungunya virus
QVIRPRPRPQRQAGQ

SEQ ID NO: 6 - protein, 15 aa, Chikungunya virus
LAQLISAVNKLTMRA

SEQ ID NO: 7 - protein, 15 aa, Chikungunya virus
SAVNKLTMRAVPQQK

SEQ ID NO: 8 - protein, 15 aa, Chikungunya virus
CMKIENDCIFEVKHE

SEQ ID NO: 9 - protein, 15 aa, Chikungunya virus
NDCIFEVKHEGKVTG

SEQ ID NO: 10 - protein, 15 aa, Chikungunya virus
EVKHEGKVTGYACLV

SEQ ID NO: 11 - protein, 15 aa, Chikungunya virus
GKVTGYACLVGDKVM

SEQ ID NO: 12 - protein, 15 aa, Chikungunya virus
YACLVGDKVMKPAHV

SEQ ID NO: 13 - protein, 15 aa, Chikungunya virus
GDKVMKPAHVKGTID

SEQ ID NO: 14 - protein, 15 aa, Chikungunya virus
KPAHVKGTIDNADLA

SEQ ID NO: 15 - protein, 15 aa, Chikungunya virus
KLAFKRSSKYDLECA

SEQ ID NO: 16 - protein, 15 aa, Chikungunya virus
EKPEGYYNWHHGAVQ

SEQ ID NO: 17 - protein, 15 aa, Chikungunya virus
YYNWHHGAVQYSGGR

SEQ ID NO: 18 - protein, 15 aa, Chikungunya virus
FTIPTGAGKPGDSGR

SEQ ID NO: 19 - protein, 15 aa, Chikungunya virus
PIFDNKGRVVAIVLG

SEQ ID NO: 20 - protein, 15 aa, Chikungunya virus
AIVLGGANEGARTAL

SEQ ID NO: 21 - protein, 15 aa, Chikungunya virus
STKDNFNVYKATRPY

SEQ ID NO: 22 - protein, 15 aa, Chikungunya virus
SHDWTKLRYMDNHMP

SEQ ID NO: 23 - protein, 15 aa, Chikungunya virus
ADAERAGLFVRTSAP

SEQ ID NO: 24 - protein, 15 aa, Chikungunya virus
TMGHFILARCPKGET

SEQ ID NO: 25 - protein, 15 aa, Chikungunya virus
THPFHHDPPVIGREK

SEQ ID NO: 26 - protein, 15 aa, Chikungunya virus
HDPPVIGREKFHSRP

SEQ ID NO: 27 - protein, 15 aa, Chikungunya virus
GNVKITVNGQTVRYK

SEQ ID NO: 28 - protein, 15 aa, Chikungunya virus
TTTDKVINNCKVDQC

SEQ ID NO: 29 - protein, 15 aa, Chikungunya virus
KVDQCHAAVTNHKKW

SEQ ID NO: 30 - protein, 15 aa, Chikungunya virus
HAAVTNHKKWQYNSP

SEQ ID NO: 31 - protein, 15 aa, Chikungunya virus
ESCKTEFASAYRAHT

SEQ ID NO: 32 - protein, 15 aa, Chikungunya virus
EFASAYRAHTASASA

SEQ ID NO: 33 - protein, 15 aa, Chikungunya virus
IVGPMSSAWTPFDNK

SEQ ID NO: 34 - protein, 15 aa, Chikungunya virus
SSAWTPFDNKIVVYK

SEQ ID NO: 35 - protein, 15 aa, Chikungunya virus
PFDNKIVVYKGDVYN

SEQ ID NO: 36 - protein, 15 aa, Chikungunya virus
IVVYKGDVYNMDYPP

SEQ ID NO: 37 - protein, 15 aa, Chikungunya virus
GDVYNMDYPPFGAGR

SEQ ID NO: 38 - protein, 15 aa, Chikungunya virus
DIQSRTPESKDVYAN

SEQ ID NO: 39 - protein, 15 aa, Chikungunya virus
TQLVLQRPAVGTVHV

SEQ ID NO: 40 - protein, 15 aa, Chikungunya virus
QRPAVGTVHVPYSQA

SEQ ID NO: 41 - protein, 15 aa, Chikungunya virus
GTVHVPYSQAPSGFK

SEQ ID NO: 42 - protein, 15 aa, Chikungunya virus
PSGFKYWLKERGASL

SEQ ID NO: 43 - protein, 15 aa, Chikungunya virus
YWLKERGASLQHTAP

SEQ ID NO: 44 - protein, 15 aa, Chikungunya virus
IPEAAFTRVVDAPSL

SEQ ID NO: 45 - protein, 15 aa, Chikungunya virus
FTRVVDAPSLTDMSC

SEQ ID NO: 46 - protein, 15 aa, Chikungunya virus
FGGVAIIKYAASKKG

SEQ ID NO: 47 - protein, 15 aa, Chikungunya virus
IIKYAASKKGKCAVH

SEQ ID NO: 48 - protein, 15 aa, Chikungunya virus
ASKKGKCAVHSMTNA

SEQ ID NO: 49 - protein, 15 aa, Chikungunya virus
KCAVHSMTNAVTIRE

SEQ ID NO: 50 - protein, 15 aa, Chikungunya virus
SMTNAVTIREAEIEV

SEQ ID NO: 51 - protein, 15 aa, Chikungunya virus
EGNSQLQISFSTALA

SEQ ID NO: 52 - protein, 15 aa, Chikungunya virus
SAEFRVQVCSTQVHC

SEQ ID NO: 53 - protein, 15 aa, Chikungunya virus
PPKDHIVNYPASHTT

SEQ ID NO: 54 - protein, 15 aa, Chikungunya virus
IVNYPASHTTLGVQD

SEQ ID NO: 55 - protein, 15 aa, Chikungunya virus
ISATAMSWVQKITGG

SEQ ID NO: 56 - protein, 15 aa, Chikungunya virus
MSWVQKITGGVGLVV

SEQ ID NO: 57 - protein, 15 aa, Chikungunya virus
NRRYQPRPWTPRSTI

SEQ ID NO: 58 - protein, 15 aa, Chikungunya virus
RPRPQRKAGQLAQLI

SEQ ID NO: 59 - protein, 15 aa, Chikungunya virus
LAQLISAVNKLTMRV

SEQ ID NO: 60 - protein, 15 aa, Chikungunya virus
SAVNKLTMRVVPQQK

SEQ ID NO: 61 - protein, 15 aa, Chikungunya virus
KPVQKKKKPGRRERM

SEQ ID NO: 62 - protein, 15 aa, Chikungunya virus
DNVMSPGYYQLLQAS

SEQ ID NO: 63 - protein, 15 aa, Chikungunya virus
MSQQSGNVKITVNSQ

SEQ ID NO: 64 - protein, 15 aa, Chikungunya virus
GNVKITVNSQTVRYK

SEQ ID NO: 65 - protein, 15 aa, Chikungunya virus
ITTDKVINNCKIDQC

SEQ ID NO: 66 - protein, 15 aa, Chikungunya virus
KIDQCHAAVTNHKKW

SEQ ID NO: 67 - protein, 15 aa, Chikungunya virus
VTHKKEIRLTVPTEG

SEQ ID NO: 68 - protein, 15 aa, Chikungunya virus
EIRLTVPTEGLEVTW

SEQ ID NO: 69 - protein, 15 aa, Chikungunya virus
LLSLLCCIRTAKAAT

SEQ ID NO: 70 - protein, 15 aa, Chikungunya virus
TQLVLQRPSAGTVHV

SEQ ID NO: 71 - protein, 15 aa, Chikungunya virus
ATNPVRAMNCAVGNM

SEQ ID NO: 72 - protein, 15 aa, Chikungunya virus
AVGNMPISIDIPDAA

SEQ ID NO: 73 - protein, 15 aa, Chikungunya virus
PISIDIPDAAFTRVV

SEQ ID NO: 74 - protein, 15 aa, Chikungunya virus
DPDSTILDIGSAPAR

SEQ ID NO: 75 - protein, 15 aa, Chikungunya virus
ILDIGSAPARRMMSD

SEQ ID NO: 76 - protein, 15 aa, Chikungunya virus
SAPARRMMSDRKYHC

SEQ ID NO: 77 - protein, 15 aa, Chikungunya virus
RMMSDRKYHCVCPMR

SEQ ID NO: 78 - protein, 15 aa, Chikungunya virus
ERLANYARKLASAAG

SEQ ID NO: 79 - protein, 15 aa, Chikungunya virus
YARKLASAAGKVLDR

SEQ ID NO: 80 - protein, 15 aa, Chikungunya virus
QVAYWVGFDTTPFMY

SEQ ID NO: 81 - protein, 15 aa, Chikungunya virus
VGFDTTPFMYNAMAG

SEQ ID NO: 82 - protein, 15 aa, Chikungunya virus
TPFMYNAMAGAYPSY

SEQ ID NO: 83 - protein, 15 aa, Chikungunya virus
AYPSYSTNWADEQVL

SEQ ID NO: 84 - protein, 15 aa, Chikungunya virus
DLTEGRRGKLSIMRG

SEQ ID NO: 85 - protein, 15 aa, Chikungunya virus
RRGKLSIMRGKKLKP

SEQ ID NO: 86 - protein, 15 aa, Chikungunya virus
SIMRGKKLKPCDRVL

SEQ ID NO: 87 - protein, 15 aa, Chikungunya virus
KKLKPCDRVLFSVGS

SEQ ID NO: 88 - protein, 15 aa, Chikungunya virus
PSVFHLKGKLSFTCR

SEQ ID NO: 89 - protein, 15 aa, Chikungunya virus
LKGKLSFTCRCDTVV

SEQ ID NO: 90 - protein, 15 aa, Chikungunya virus
CDTVVSCEGYVVKRI

SEQ ID NO: 91 - protein, 15 aa, Chikungunya virus
TMSPGLYGKTTGYAV

SEQ ID NO: 92 - protein, 15 aa, Chikungunya virus
PTDHVVGEYLVLSPQ

SEQ ID NO: 93 - protein, 15 aa, Chikungunya virus
LTNPPYHEFAYEGLK

SEQ ID NO: 94 - protein, 15 aa, Chikungunya virus
YHEFAYEGLKIRPAC

SEQ ID NO: 95 - protein, 15 aa, Chikungunya virus
KGKWNINKQICVTTR

SEQ ID NO: 96 - protein, 15 aa, Chikungunya virus
GERMEWLVNKINGHH

SEQ ID NO: 97 - protein, 15 aa, Chikungunya virus
WLVNKINGHHVLLVS

SEQ ID NO: 98 - protein, 15 aa, Chikungunya virus
LGRYDLVVINIHTPF

SEQ ID NO: 99 - protein, 15 aa, Chikungunya virus
RVICVLGRKFRSSRA

SEQ ID NO: 100 - protein, 15 aa, Chikungunya virus
SYRVKRMDIAKNDEE

SEQ ID NO: 101 - protein, 15 aa, Chikungunya virus
CVVNAANPRGLPGDG

SEQ ID NO: 102 - protein, 15 aa, Chikungunya virus
VCKAVYKKWPESFKN

SEQ ID NO: 103 - protein, 15 aa, Chikungunya virus
ESFKNSATPVGTAKT

SEQ ID NO: 104 - protein, 15 aa, Chikungunya virus
YPVIHAVGPNFSNYS

SEQ ID NO: 105 - protein, 15 aa, Chikungunya virus
AYREVAKEVTRLGVN

SEQ ID NO: 106 - protein, 15 aa, Chikungunya virus
AKEVTRLGVNSVAIP

SEQ ID NO: 107 - protein, 15 aa, Chikungunya virus
WEKKISEAIQMRTQV

SEQ ID NO: 108 - protein, 15 aa, Chikungunya virus
QTEANEQVCLYALGE

SEQ ID NO: 109 - protein, 15 aa, Chikungunya virus
RQKCPVDDADASSPP

SEQ ID NO: 110 - protein, 15 aa, Chikungunya virus
PKYKIEGVQKVKCSK

SEQ ID NO: 111 - protein, 15 aa, Chikungunya virus
EGVQKVKCSKVMLFD

SEQ ID NO: 112 - protein, 15 aa, Chikungunya virus
VKCSKVMLFDHNVPS

SEQ ID NO: 113 - protein, 15 aa, Chikungunya virus
VMLFDHNVPSRVSPR

SEQ ID NO: 114 - protein, 15 aa, Chikungunya virus
HNVPSRVSPREYRSS

SEQ ID NO: 115 - protein, 15 aa, Chikungunya virus
AVSDWVMSTVPVAPP

SEQ ID NO: 116 - protein, 15 aa, Chikungunya virus
PRGAIKVTAQLTDHV

SEQ ID NO: 117 - protein, 14 aa, Chikungunya virus
VTAQLTDHVVGEYL

SEQ ID NO: 118 - protein, 15 aa, Chikungunya virus
LTDHVVGEYLVLSPQ

SEQ ID NO: 119 - protein, 15 aa, Chikungunya virus
QESVREVSMTTSLTH

SEQ ID NO: 120 - protein, 15 aa, Chikungunya virus
HPPISFGAPSETFPI

SEQ ID NO: 121 - protein, 15 aa, Chikungunya virus
NDGEIESLSSELLTF

SEQ ID NO: 122 - protein, 15 aa, Chikungunya virus
PRGAIKVTAQPTDHV

SEQ ID NO: 123 - protein, 15 aa, Chikungunya virus
KVTAQPTDHVVGEYL

SEQ ID NO: 124 - protein, 15 aa, Chikungunya virus
FSNYSESEGDRELAA

SEQ ID NO: 125 - protein, 15 aa, Chikungunya virus
HPPISFGASSETFPI

SEQ ID NO: 126 - protein, 15 aa, Chikungunya virus
NEGEIESLSSELLTF

SEQ ID NO: 127 - protein, 15 aa, Chikungunya virus
RPRPQRQAGQLAQLI

SEQ ID NO: 128 - protein, 15 aa, Chikungunya virus
KPAQKKKKPGRRERM

SEQ ID NO: 129 - protein, 15 aa, Chikungunya virus
DNVMRPGYYQLLQAS

SEQ ID NO: 130 - protein, 15 aa, Chikungunya virus
MSQQSGNVKITVNGQ

SEQ ID NO: 131 - protein, 15 aa, Chikungunya virus
VMHKKEVVLTVPTEG

SEQ ID NO: 132 - protein, 15 aa, Chikungunya virus
EVVLTVPTEGLEVTW

SEQ ID NO: 133 - protein, 15 aa, Chikungunya virus
LLSLICCIRTAKAAT

SEQ ID NO: 134 - protein, 15 aa, Chikungunya virus
EQQPLFWLQALIPLA

SEQ ID NO: 135 - protein, 15 aa, Chikungunya virus
ATNPVRAVNCAVGNM

SEQ ID NO: 136 - protein, 15 aa, Chikungunya virus
AVGNMPISIDIPEAA

SEQ ID NO: 137 - protein, 15 aa, Chikungunya virus
PISIDIPEAAFTRVV

SEQ ID NO: 138 - protein, 15 aa, Chikungunya virus
KVTAQLTDHVVGEYL

SEQ ID NO: 139 - protein, 15 aa, Chikungunya virus
FSNYTESEGDRELAA

SEQ ID NO: 140 - protein, 15 aa, Chikungunya virus
EQQPLFWMQALIPLA

SEQ ID NO: 141 - protein, 15 aa, Chikungunya virus
FNVYKATRPYLAHCP

SEQ ID NO: 142 - protein, 15 aa, Chikungunya virus
SPYVKCCGTAECKDK

SEQ ID NO: 143 - protein, 15 aa, Chikungunya virus
QSLNHLFTAMDSTDA

SEQ ID NO: 144 - protein, 15 aa, Chikungunya virus
ILYYYELYPTMTVVV

SEQ ID NO: 145 - protein, 15 aa, Chikungunya virus
ALIVLCNCLRLLPCC

SEQ ID NO: 146 - protein, 15 aa, Chikungunya virus
CARRRCITPYELTPG

SEQ ID NO: 147 - protein, 15 aa, Chikungunya virus
FFNMMQMKVNYNHNI

SEQ ID NO: 148 - protein, 10 aa, Chikungunya virus
KLSIMRGKKL

SEQ ID NO: 149 - protein, 9 aa, Chikungunya virus
IKYAASKKG

SEQ ID NO: 150 - protein, 11 aa, Chikungunya virus
LISAVNKLTMR